(19) United States
(12) Patent Application Publication
(10) Pub. No.: US 2009/0286827 A1
(43) Pub. Date: Nov. 19, 2009

(54) NOVEL BI-ARYL AMINES

(76) Inventors: Ralf Glatthar, Bad Sackingen (DE); David Carcache, Binninggen (CH); Carsten Spanka, Lorrach (DE); Ivan-toma Vranesic, basel (CH); Thomas J. Troxler, Wahlen b. Laufen (CH)

Correspondence Address: FOLEY AND LARDNER LLP;SUITE 500, 3000 K STREET NW, WASHINGTON, DC, 20007, US

(21) Appl. No.: 12/296,034

(22) PCT Filed: Apr. 2, 2007
(86) PCT No.: PCT/EP07/53155

§ 371 Date: Oct. 3, 2008

(30) Foreign Application Priority Data
Apr. 3, 2006 (GB) 0606774.8

(51) Int. Cl. A61K 31/437; C07D 401/04; C07D 471/04; C07D 487/00; A61K 31/4439; A61K 31/4162

(52) U.S. Cl. 514/303; 546/274.1; 546/119; 546/121; 546/268.4; 540/473; 514/341; 514/381

(57) ABSTRACT

The present invention relates to novel bi-aryl amines of formula (I) and to pharmaceutically acceptable prodrugs, salts, solvates, hydrates, and N-oxides thereof and to pharmaceutical compositions comprising them, methods of their use, and methods of their preparation.

The present invention relates to novel compounds, their preparation, their use as pharmaceuticals and pharmaceutical compositions containing them.

WO2005/079802 describes bipyridylamides and their use as modulators of metabotrobic glutamate receptor-5. The compounds show valuable properties, but also have disadvantages. Thus, there is a need to provide further compounds having properties as modulators of metabotrobic glutamate receptor-5.

In a first aspect, the invention relates to a compound of formula

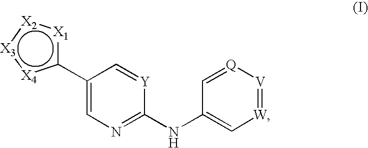

wherein
(i) X₁, X₂, X₃, and X₄ are independently selected from the group consisting of CR¹, CO, N, NR², O and S,
(ii) R¹ and R² are independently selected from the group consisting of H, alkyl, substituted alkyl, benzyl, substituted benzyl, phenyl and substituted phenyl, or R₁ and R₂ form together with the atoms to which they are attached a hydrocarboncycle, a substituted hydrocarboncycle, a heterocycle or a substituted heterocycle,
(iii) Y represents CH or CR³ or N
(iv) V represents CH, CR⁴ or N
(v) Q represents CH, CR⁵ or N
(vi) W represents CH, CR⁶ or N, and
(vii) R³, R⁴, R⁵, and R⁶ are independently selected from the group consisting of OH, halogen, alkyl, trifluoralkyl, alkoxy, trifluoralkoxy, and CN;
   and pharmaceutically acceptable prodrugs, salts, solvates, hydrates, and N-oxides thereof.

More precisely, the invention relates to new compounds of formula

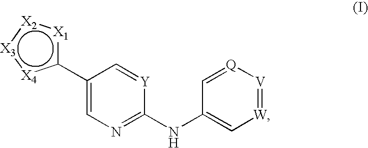

wherein
(i) the five member ring has 6 π-electrons with the proviso that the C-atom and three of the moieties of X1, X2, X3, X4 contribute each 1 n-electron and one moiety of X1, X2, X3, X4 contribute 2 π-electrons to the 6 π-electrons of the five member ring,
(ii) X₁, X₂, X₃, and X₄ are independently selected from the group consisting of CR¹, CO, N, NR², O and S,
(iii) R¹ and R² are independently selected from the group consisting of H, alkyl, substituted alkyl, benzyl, substituted benzyl, phenyl and substituted phenyl, or R₁ and R₂ form together with the atoms to which they are attached a hydrocarbon cycle, a substituted hydrocarbon cycle, a heterocycle or a substituted heterocycle,
(iv) Y represents CH or CR³ or N
(v) V represents CH, CR⁴ or N
(vi) Q represents CH, CR⁵ or N
(vii) W represents CH, CR⁶ or N, and
(viii) R³, R⁴, R⁵, and R⁶ are independently selected from the group consisting of OH, halogen, alkyl, trifluoralkyl, alkoxy, trifluoralkoxy, and CN;
   and pharmaceutically acceptable prodrugs, salts, solvates, hydrates, and N-oxides thereof.

The following information relates to both aspects (first and second aspect of the invention) as defined above. Accordingly, some of the compounds of the formula (I) may exist in two or more tautomeric forms. The skilled person will recognise that the particular tautomeric form and/or the proportion of different tautomeric forms in which a compound of the invention exists may vary depending on the conditions to which the compound is subjected. All such tautomeric forms as well as mixtures thereof are part of the present invention.

Compounds of formula (I) exist in free or acid addition salt form. In this specification, unless otherwise indicated, language such as “compounds of formula (I)” is to be understood as embracing the compounds in any form, for example free base or acid addition salt form. Salts which are unsuitable for pharmaceutical uses but which can be employed, for example, for the isolation or purification of free compounds of formula (I), such as picrates or perchlorates, are also included. For therapeutic use, only pharmaceutically acceptable salts or free compounds are employed (where applicable in the form of pharmaceutical preparations), and are therefore preferred.

In the present specification, the following definitions shall apply if no specific other definition is given:

“Alkyl” represents a straight-chain or branched-chain alkyl group, preferably represents a straight-chain or branched-chain C₁₋₁₂alkyl, particularly preferably represents a straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl.

The term “cycloalkyl” refers to optionally substituted monocyclic, bicyclic or tricyclic hydrocarbon groups of 3-12 carbon atoms, each of which may contain one or more carbon to carbon double bonds, or the cycloalkyl may be substituted by one or more substituents, such as alkyl, halo, oxo, hydroxy, alkoxy, alkanoyl, acylamino, carbamoyl, alkylamino, dialkylamino, thiol, alkylthio, cyano, carboxy, alkoxycarbonyl, sulfonyl, sulfonamido, sulfamoyl, heterocyclyl and the like.

Exemplary monocyclic hydrocarbon groups include, but are not limited to, cyclopropyl, cyclopropylmethyl, cyclobutyl, cyclopentyl, cyclopentenyl, cyclohexyl and cyclohexenyl and the like.

“Alkandiyl” represents a straight-chain or branched-chain alkandiyl group bound by two different Carbon atoms to the molecule, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

Each alkyl part of “alkoxy”, “alkoxyalkyl”, “alkoxycarbonyl”, “alkoxycarbonylalkyl” and “halogenalkyl” shall have the same meaning as described in the above-mentioned definition of “alkyl”.

“Alkenyl” represents a straight-chain or branched-chain alkenyl group, preferably C₂₋₆alkenyl, for example, vinyl, allyl, 1-propenyl, isopropenyl, 2-butenyl, 2-pentenyl, 2-hexenyl, etc. and preferably represents C₂₋₄alkenyl.

“Alkendiyl” represents a straight-chain or branched-chain alkendiyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—.

“Alkynyl” represents a straight-chain or branched-chain alkynyl group, preferably C₂₋₆alkynyl, for example, ethenyl, propargyl, 1-propynyl, isopropenyl, 1-(2- or 3) butynyl, 1-(2- or 3) pentenyl, 1-(2- or 3) hexenyl, etc., preferably represents C₂₋₄alkynyl and particularly preferably represents ethynyl.

“Aryl” represents an aromatic hydrocarbon group, preferably a C₆₋₁₀ aromatic hydrocarbon group; for example phenyl, naphthyl, especially phenyl.

“Aralkyl” denotes an “Aryl” bound to an “Alkyl” (both as defined above) an represents, for example benzyl, α-methylbenzyl, 2-phenylethyl, α,α-dimethylbenzyl, especially benzyl.

“Heterocycle” represents a saturated, partly saturated or aromatic ring system containing at least one hetero atom. Preferably, heterocycles consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Oxo (═O), Halogen, Nitro, Cyano, Alkyl, Alkandiyl, Alkenediyl, Alkoxy, Alkoxyalkyl, Alkoxycarbonyl, Alkoxycarbonylalkyl, Halogenalkyl, Aryl, Aryloxy, Arylalkyl. Examples of heterocyclic moieties are: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazlolidine, isothiazole, istothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline and the like.

“Hetero atoms” are atoms other than Carbon and Hydrogen, preferably Nitrogen (N), Oxygen (O) or Sulfur (S).

“Halogen” represents Fluoro, Chloro, Bromo or Iodo, preferably represents Fluoro, Chloro or Bromo and particularly preferably represents Chloro.

Preferred substituents, preferred ranges of numerical values or preferred ranges of the radicals present in the formula (I) and the corresponding intermediate compounds are defined below.

Preferably one of the moieties X₁, X₂, X₃, and X₄ represents N, another one of the moieties X₁, X₂, X₃, and X₄ represents NR², a further one of the moieties X₁, X₂, X₃, and X₄ represents CR¹ and the remaining one of the moieties X₁, X₂, X₃, and X₄ represents either CH or N. More preferably X₁ represents N. Still more preferably X₄ represents NR². Yet more preferably X₃ represents CR¹ and X₂ represents CR¹ or N. In a preferred embodiment the moieties X₁, X₂, X₃, and X₄ are defined as follows: X₁ represents N, X₂ is CH, X₃ is CH or CCH₃, and X₄ is NR² with R² being a C₁ to C₄ alkyl, and optionally R₁ and R₂ form together with the atoms to which they are attached a six member ring.
R¹ preferably represents H, straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl.
R² preferably represents straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl. Moreover R represents preferably cyclohexyl or cyclopropylmethyl.
R³ preferably represents halogen or alkyl.
R⁴ preferably represents halogen or alkyl.
R⁵ particularly preferably represents alkyl.
Y preferably represents CH or CR³
Y particularly preferably represents CH or CCl.
Q preferably represents CH or N.
W preferably represents CH.
V preferably represents CCl or CCH₃.

In a preferred embodiment R¹ and R² form together with the Nitrogen atom to which R² is attached and with the carbon atom to which R¹ is attached an unsubstituted or substituted heterocycle having 3-11 ring atoms and 1-4 hetero atoms; the hetero atoms being selected from the group consisting of N, O, S, the substituents being selected from the group consisting of Oxo (═O), Hydroxy, Halogen, Amino, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl. More preferably the R¹ and R² form together with the Nitrogen atom at position X₄ to which R² is attached and with the carbon atom at position X₃ to which R¹ is attached an unsubstituted heterocycle having 6 ring atoms and one nitrogen.

The abovementioned general or preferred radical definitions apply both to the end products of the formula (I) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formula (I) which contain a combination of the meanings mentioned above as being preferred.

Particular preference according to the invention is given to compounds of the formula (I) which contain a combination of the meanings listed above as being particularly preferred.

More particular preference according to the invention is given to the compounds of the formula (I) which contain a combination of the meanings listed above as being very particularly preferred.

Still more preferred compounds are selected from the group consisting of

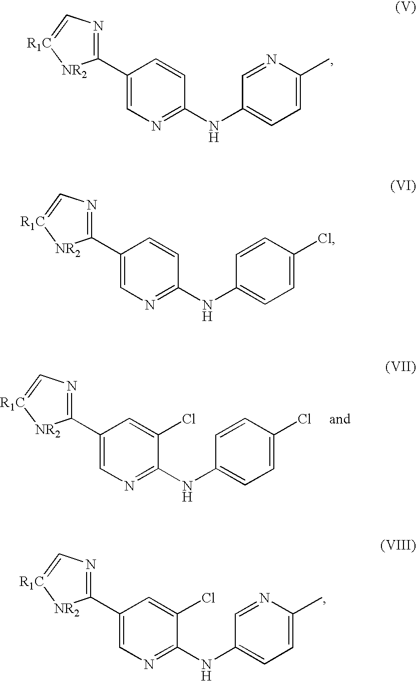

wherein R¹ represents H or CH₃ and R² represents CH₃, ethyl, n-propyl, isopropyl, isopropylmethyl, cyclopropylmethyl, cyclohexyl, phenyl and benzyl.

Particular preferred compounds of formula (I) are the following:

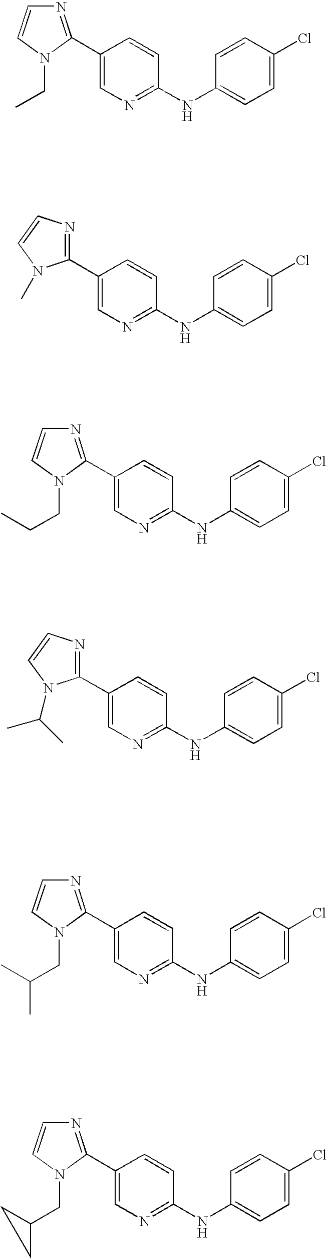

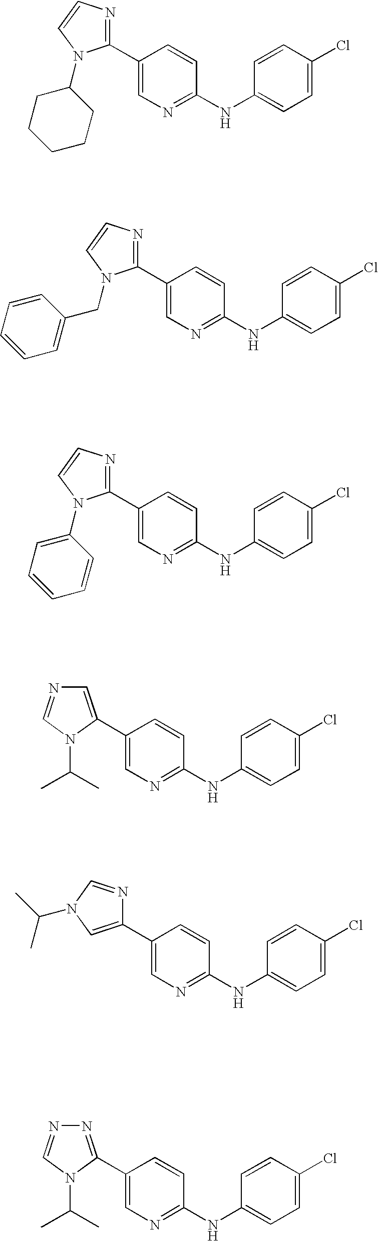

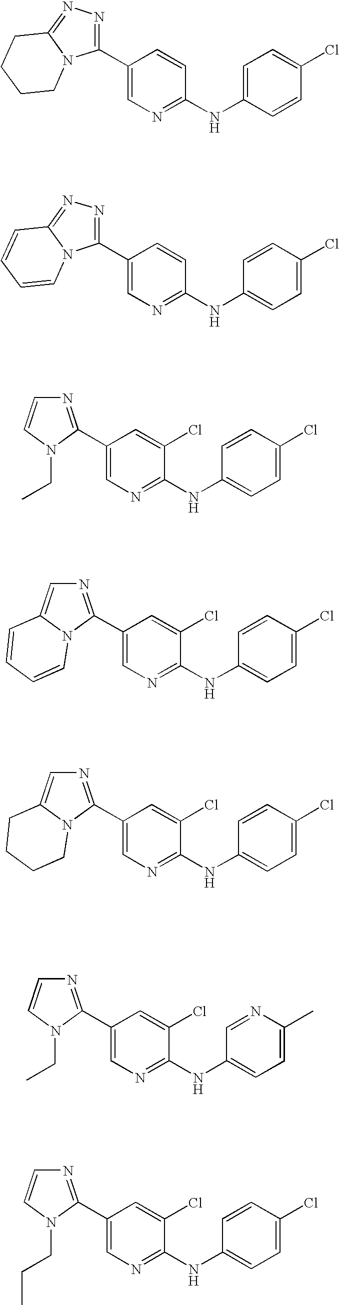

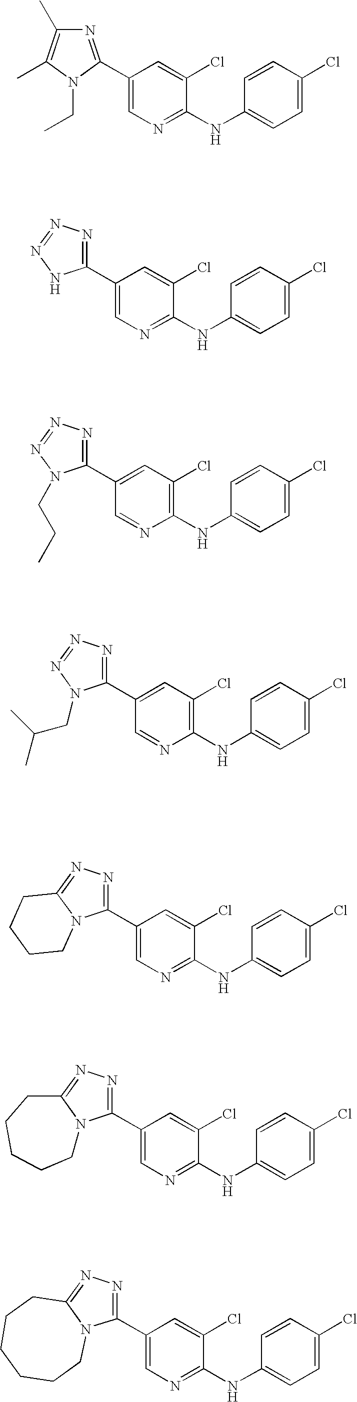

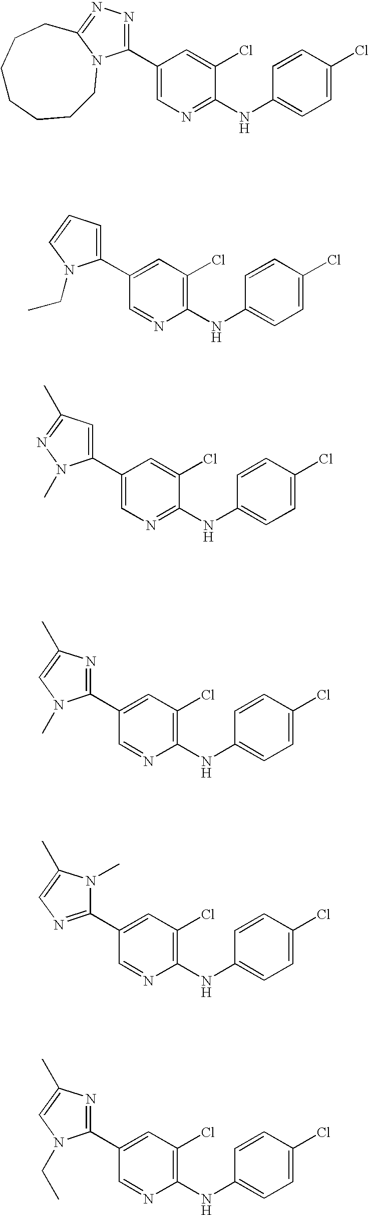

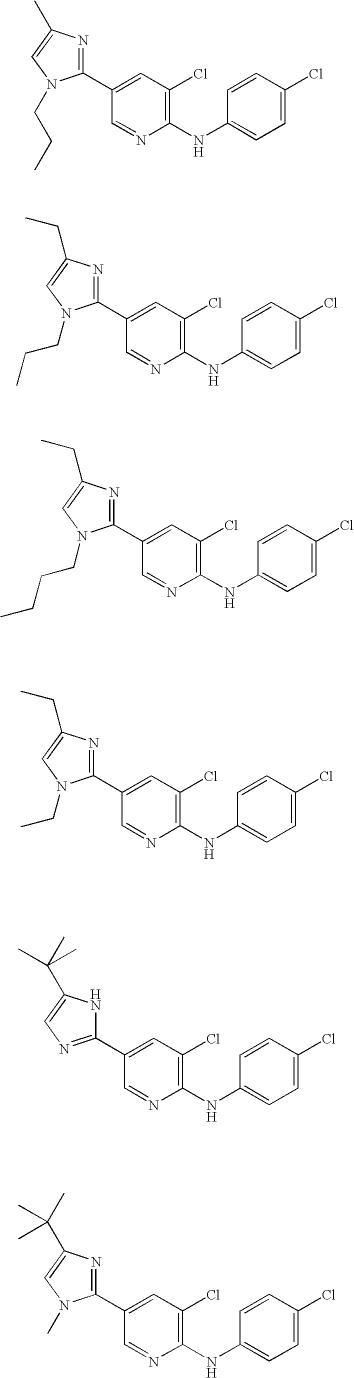

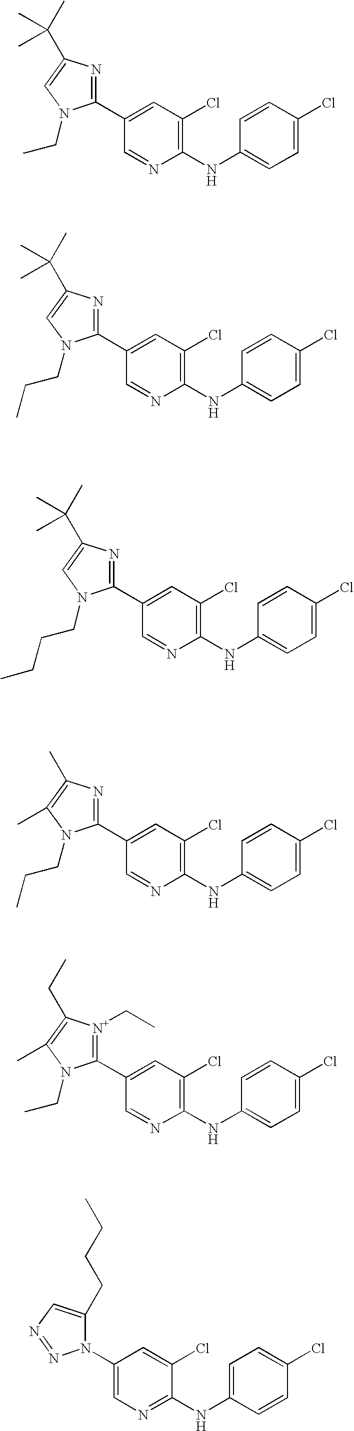

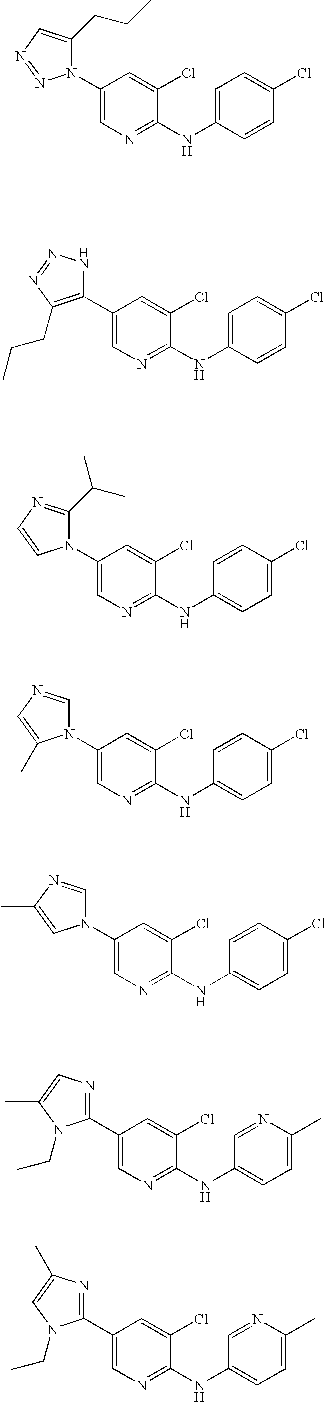

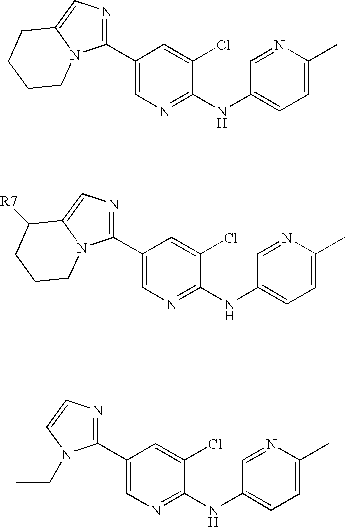

wherein R⁷ is alkyl or aryl as defined above;
including pharmaceutically acceptable prodrugs, salts, solvates, hydrates, and N-oxides thereof.

Particular preferred compounds of formula (I) are the following:

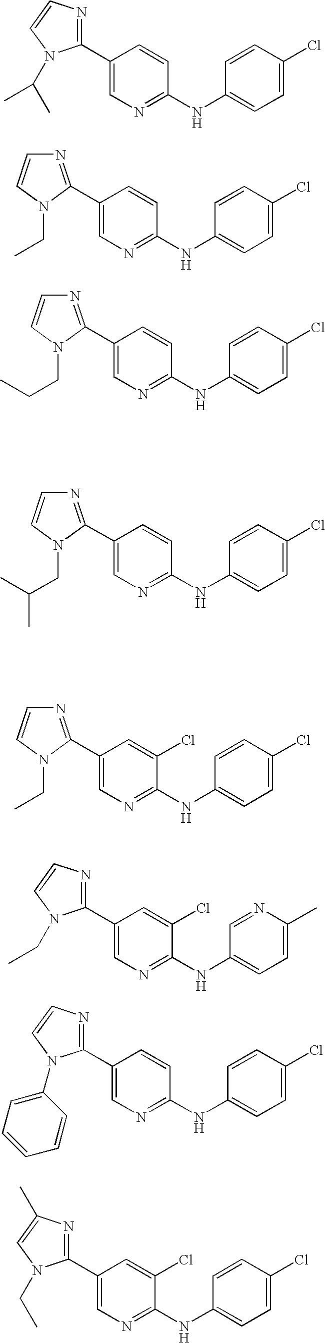

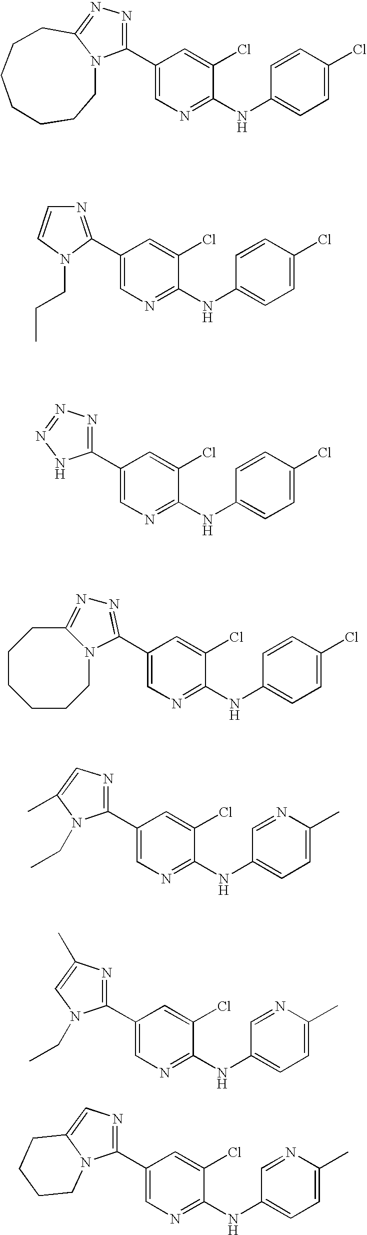

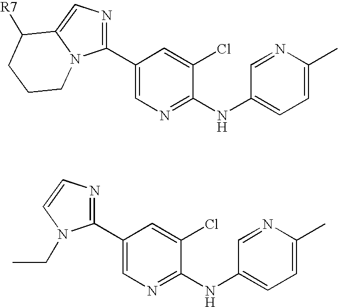

wherein R⁷ is alkyl or aryl as defined above;
including pharmaceutically acceptable prodrugs, salts, solvates, hydrates, and N-oxides thereof.

In a further aspect, the invention provides process for the production of the compounds of formula (I) and their salts as defined above.

The process comprises at least one of the steps (A), (B) or (C) as defined below.

The process step (A) is as follows:

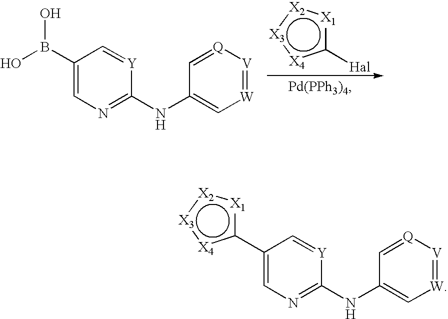

Preferably in step (A) additionally Na₂CO₃, methanol and inert solvent, more preferably benzene is used. As a preferred halogen (Hal) brome is used.

Process step (B) is as follows:

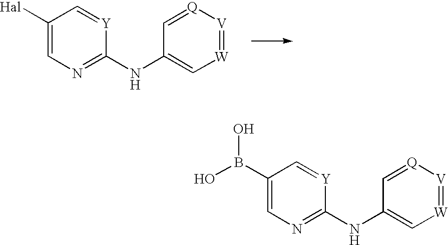

It is preferred that step (B) takes place in the presence of B(Oalkyl)₃, more preferred B(OiPr)₃, and BuLi in hexane. Preferably step (B) takes place in advance of step (A).

Process step (C) is as follows:

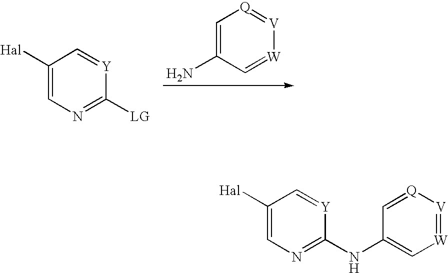

wherein LG represents a leaving group such as bromine, chlorine, fluorine, methoxy, preferably chlorine, and the other moieties Y, Q, V, W are as defined above and optionally the step (C) takes place in the presence of a reaction auxiliary, as NaH, and recovering the resulting compound in free base or acid addition salt form. The starting materials of step (C) are known or obtainable according to known methods

Preferably step (C) takes place in advance of step (A) or step (B).

Even more preferred the process steps (A), (B), (C) takes place in the order of (C)→(B)→(A).

Still more preferred the moieties in the formulae given in the steps (A), (B) and (C) are the same as defined for the formula (I), in particular the moieties are as follows:
(i) Y is CH or CCl
(ii) Q is CH or N
(iii) W is CH
(iv) V is CCl or CCH₃, and
(v) one of the moieties X₁, X₂, X₃, and X₄ is N, another one of the moieties X₁, X₂, X₃, and X₄ is NR², a further one of the moieties X₁, X₂, X₃, and X₄ is CR¹ and the remaining one of the moieties X₁, X₂, X₃, and X₄ is either CH or N.

The following considerations apply to the individual reaction steps described above:

a) One or more functional groups, for example carboxy, hydroxy, amino, or mercapto, may need to be protected in the starting materials by protecting groups. The protecting groups employed may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.
b) Acid addition salts may be produced from the free bases in known manner, and vice-versa. Compounds of formula (I) in optically pure form can be obtained from the corresponding racemates according to well-known procedures, e.g. HPLC with chiral matrix. Alternatively, optically pure starting materials can be used.
c) Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar pro-cedures. This separation may take place either at the level of a starting compound or in a compound of formula I itself. Enantiomers may be separated through the formation of dia-stereomeric salts, for example by salt formation with an enantiomer-pure chiral acid, or by means of chromatography, for example by HPLC, using chromatographic substrates with chiral ligands.
d) Suitable diluents for carrying out the above-described are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethyelene glycol monomethyl ether, diethylene glycol monoethyl ether. Further, mixtures of diluents may be employed. Depending on the starting materials, reaction conditions and auxiliaries, water or diluents containing water may be suitable. It is also possible to use one a starting material as diluent simultaneously.
e) Reaction temperatures can be varied within a relatively wide range. In general, the processes are carried out at temperatures between 0° C. and 150° C., preferably between 10° C. and 120° C. Deprotonation reactions can be varied within a relatively wide range. In general, the processes are carried out at temperatures between −150° C. and +50° C., preferably between −75° C. and 0° C.
f) The reactions are generally carried out under atmospheric pressure. However, it is also possible to carry out the processes according to the invention under elevated or reduced pressure—in general between 0.1 bar and 10 bar.
g) Starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours.
h) Work-up is carried out by customary methods (cf. the Preparation Examples).
i) A compound of formula (I) obtained according to the above described processes can be converted into another compound of formula (I) according to conventional methods.

Compounds of formulae (I) (as defined above), (II), (III), (IV) and their pharmaceutically acceptable acid addition salts, hereinafter referred to as agents of the invention, exhibit valuable pharmacological properties and are therefore useful as pharmaceuticals.

In particular, the agents of the invention exhibit a marked and selective modulating, especially antagonistic, action at human metabotropic glutamate receptors (mGluRs). This can be determined in vitro for example at recombinant human metabotropic glutamate receptors, especially PLC-coupled subtypes thereof such as mGluR5, using different procedures like, for example, measurement of the inhibition of the agonist induced elevation of intracellular Ca²⁺ concentration in accordance with L. P. Daggett et al., Neuropharm. Vol. 34, pages 871-886 (1995), P. J. Flor et al., J. Neurochem. Vol. 67, pages 58-63 (1996) or by determination to what extent the agonist induced elevation of the inositol phosphate turnover is inhibited as described by T. Knoepfel et al., Eur. J. Pharmacol. Vol. 288, pages 389-392 (1995), L. P. Daggett et al., Neuropharm. Vol. 34, pages 871-886 (1995) and references cited therein. Isolation and expression of human mGluR subtypes are described in U.S. Pat. No. 5,521,297. Selected agents of the invention show IC50 values for the inhibition of the agonist (e.g. glutamate or quisqualate) induced elevation of intracellular Ca2+ concentration or the agonist (e.g. glutamate or quisqualate) induced inositol phosphate turnover, measured in recombinant cells expressing hmGluR5a of about 1 nM to about 50 μM.

The agents of the invention are therefore useful in the treatment of disorders associated with irregularities of the glutamatergic signal transmission, and of nervous system disorders mediated full or in part by mGluR5.

The agents of the invention are therefore useful in the prevention, treatment or delay of progression of disorders associated with irregularities of the glutamatergic signal transmission, of the gastrointestinal and urinary tract and of nervous system disorders mediated full or in part by mGluR5.

Disorders associated with irregularities of the glutamatergic signal transmission are for example epileptogenesis including neuronal protection after status epilepticus, cerebral ischemias, especially acute ischemias, ischemic diseases of the eye, muscle spasms such as local or general spasticity, skin disorders, obesity disorders, and, in particular, convulsions or pain.

Disorders of the gastrointestinal tract include Gastro-Esophageal Reflux Disease (GERD), Functional Gastro-intestinal Disorders and Post-operative Ileus.

Functional Gastro-intestinal Disorders (FGIDs) are defined as chronic or recurrent conditions associated with abdominal symptoms without organic cause using conventional diagnostic measures. A cardinal symptom present in many FGIDs is visceral pain and/or discomfort. FGIDs include functional dyspepsia (FD), functional heartburn (a subset of GERD), irritable bowel syndrome (IBS), functional bloating, functional diarrhea, chronic constipation, functional disturbancies of the biliary tract as well as other conditions according to Gut 1999; Vol. 45 Suppl. II.

Post-operative Ileus is defined as failure of aboral passage of intestinal contents due to transient impairment of GI motility following abdominal surgery.

Disorders of the Urinary Tract comprise conditions associated with functional disturbancies and/or discomfort/pain of the urinary tract. Examples of disorders of the urinary tract include but are not limited to incontinence, benign prostatic hyperplasia, prostatitis, detrusor hyperreflexia, outlet obstruction, urinary frequency, nocturia, urinary urgency, overactive bladder (OAB), pelvic hypersensitivity, urge incontinence, urethritis, prostatodynia, cystitis, idiopathic bladder hypersensitivity and the like. OAB is a syndrome characterized by urgency, with or without urinary incontinence, and usually with increased voiding frequency and nocturia.

Inflammatory diseases, such as pain, inflammation and/or oedema consequential to trauma, for example associated with burns, sprains, fractures or the like, inflammatory airways diseases, such as COPD, asthma, rhinitis, inflammatory bowel disease, cystitis, uveitis, inflammatory skin disorders, such as psoriasis or eczema, rheumatoid arthritis, use as a smooth muscle relaxant, for example for the treatment of spasms of the gastrointestinal tract or uterus, for example in the therapy of Crohn's disease, ulcerative collitis or pancreatitis, or for the treatment of muscle spasticity and tremor, for example in multiple sclerosis, teno-synovitis, gout, ocular disorders, for example glaucoma, cough.

Nervous system disorders mediated full or in part by mGluR5 are for example acute, traumatic and chronic degenerative processes of the nervous system, such as Parkinson's disease, Parkinson's dyskinesia, senile dementia, Alzheimer's disease, Huntington's chorea, amyotrophic lateral sclerosis, multiple sclerosis and fragile X syndrome, substance-related disorders, psychiatric diseases such as schizophrenia, affective and anxiety disorders, attention deficit disorders and cognitive dysfunction associated with these and other CNS disorders. Substance-related disorders include substance abuse, substance dependence and substance withdrawal disorders, e.g. nicotine withdrawal. Anxiety disorders includes panic disorder, social and specific phobias, anxiety, obsessive compulsive disorder (OCD), post traumatic stress disorder (PTSD) and generalized anxiety disorder (GAD). Affective disorders include depressive (major depression, dysthymia, depressive disorders NOS) and bipolar disorders (bipolar I and II disorders). Cognitive dysfunction associated with these and other CNS disorders include deficits and abnormalities in attention and vigilance, executive functions and memory (for instance working memory and episodic memory). Other disorders which are mediated fully or in part are pain and itch.

A further disorder is migraine.

The compounds and compositions of the present invention may also be useful for treating cognitive impairment and/or attention deficit disorder.

Cognitive dysfunction include deficits and abnormalities in attention and vigilance, executive functions and memory (for instance working memory and episodic memory). Other disorders relating to cognitive dysfunction include sleep related breathing disorders (SRBD), behavioral impairments, information processing deficits and age-related disorders.

Further examples falling of cognitive impairment and/or attention deficit disorders include: Attention-deficit hyperactivity disorder (ADHD), childhood ADHD, adult ADHD, excess daytime somnolence, sleep apnea, shift-worker's sleep-wake cycle disruption, traumatic brain injury, neurodegenerative disorders with associated memory and cognitive problems (such as Alzheimer's disease, Lewy body dementia, senile dementia, vascular dementia, Parkinson's disease), chronic fatigue syndrome, fatigue associated with sleep deprivation or prolonged wakefulness, age-related decline in memory and cognitive function (such as mild cognitive impairment), cognitive impairment associated with mood disorders (such as depression) and anxiety, schizophrenia, day time sleepiness associated with narcolepsy.

Furthermore, the compounds of the present invention may provide treatment for or improve of the cognitive enhancement of a subject. The term “cognitive enhancement” includes, but is not limited to, cognition enhancement, vigilance, counteracting effects of fatigue, enhancing alertness, attention, memory (working, episodic), learning ability, reaction time, cognitive performance enhancement, excess daytime somnolence, reversal of information processing deficits, improvement of disorganization, i.e. improving organizational skills/level of organizational ability.

The compounds and compositions of the present invention may also be useful for the delay of progression of the above-mentioned conditions and disorders.

The usefulness of the agents of the invention in the treatment of the above-mentioned disorders can be confirmed in a range of standard tests including those indicated below:

Activity of the agents of the invention in anxiety can be demonstrated in standard models such as the stress-induced hyperthermia in mice [cf. A. Lecci et al., Psychopharmacol. 101, 255-261]. At doses of about 0.1 to about 30 mg/kg p.o., selected agents of the invention reverse the stress-induced hyperthermia.

At doses of about 4 to about 50 mg/kg p.o., selected agents of the invention show reversal of Freund complete adjuvant (FCA) induced hyperalgesia [cf. J. Donnerer et al., Neuroscience 49, 693-698 (1992) and C. J. Woolf, Neuroscience 62, 327-331 (1994)].

Activity of the agents of the invention in GERD can be demonstrated in standard models such as the gastric distension-induced transient lower esophageal sphincter relaxations (TLESRs) in dogs. At doses of about 0.03 to about 10 mg/kg p.o., selected agents of the invention reduce the occurrence of TLESRs.

Activity of the agents of the invention in functional dyspepsia can be demonstrated a model of fasted gastric tone and gastric accommodation to meal in dogs. At doses of about 0.03 to about 10 mg/kg p.o., selected agents of the invention increase the gastric volume in fasting conditions indicative of a reduced gastric tone.

Activity of the agents of the invention in visceral hyperalgesia can be demonstrated in standard rat models according to modified methods by Tarrerias, A. et al., Pain (2002) 100: 91-97, Schwetz, I. et al., Am. J. Physiol. (2005) 286: G683-G691, of La, J. et al., World J. Gastroenterol. (2003) 9: 2791-2795. At doses of about 0.03 to about 30 mg/kg p.o., selected agents of the invention reduce the exaggerated abdominal striated muscle contractions, indicative of a visceral antinociceptive activity.

Activity of the agents of the invention in visceral sensation/pain of the urinary bladder can be demonstrated in a standard mouse model according to a modified method by Ness T J and Elhefni H. J Urol. (2004) 171:1704-8. At doses of about 0.3 to about 30 mg/kg p.o., selected agents of the invention reduce the EMG (visceromotor) response, indicative of a visceral antinociceptive and/or hyposensitivity.

Activity of the agents of the invention in overactive bladder and urge incontinence can be demonstrated in standard cystometry models in rats according to modified method by Tagaki-Matzumoto et al J. Pharmacol. Sci. (2004) 95: 458-465. At doses of about 0.03 to about 10 mg/kg p.o., selected agents of the invention increased threshold volumes eliciting bladder contractions indicative of therapeutic potential in conditions with bladder dysfunctions.

For all the above mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.05 to about 100 mg/kg animal body weight. In larger mammals, for example humans, an indicated daily dosage is in the range from about 5 to 1500 mg, preferably about 10 to about 1000 mg of the compound conveniently administered in divided doses up to 4 times a day or in sustained release form.

In accordance with the foregoing, the present invention also provides in a further aspect an agent of the invention for use as a pharmaceutical, e.g. in the treatment of disorders associated with irregularities of the glutamatergic signal transmission, and of nervous system disorders mediated full or in part by mGluR5.

The invention also provides the use of an agent of the invention, in the treatment of disorders associated with irregularities of the glutamatergic signal transmission, and of nervous system disorders mediated full or in part by mGluR5.

In a further aspect, the invention provides the use of compounds of formula (I) as modulators of metabotrobic Glutamate Receptors, Subtype 5 (“mGluR5-Modulators”).

Furthermore the invention provides the use of an agent of the invention for the manufacture of a pharmaceutical composition designed for the treatment of disorders associated with irregularities of the glutamatergic signal transmission, and of nervous system disorders mediated full or in part by mGluR5.

In a further aspect the invention relates to a method of treating disorders mediated full or in part by mGluR5, which method comprises administering to a warm-blooded organism in need of such treatment a therapeutically effective amount of an agent of the invention.

Moreover the invention relates to a pharmaceutical composition comprising an agent of the invention in association with one or more pharmaceutical carrier or one or more pharmaceutically acceptable diluent.

The pharmaceutical compositions according to the invention are compositions for enteral, such as nasal, rectal or oral, or parenteral, such as intramuscular or intravenous, administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Preferred are the compounds according to the examples.

Further, properly isotope-labeled agents of the invention exhibit valuable properties as histopathological labeling agents, imaging agents and/or biomarkers, hereinafter “markers”, for the selective labeling of mGluR5. More particularly the agents of the invention are useful as markers for labeling the central and peripheral mGlu5 receptors in vitro or in vivo. In particular, compounds of the invention which are properly isotopically labeled are useful as ligands to image mGlu5 receptors in vivo or in vitro studies. Suitable radionuclides that may be incorporated in the agents of invention include: 3H, 11C, 13N, 15O, 18F, 123I, 125I, 131I, 75Br, 76Br, 77Br, 82Br, 99mTc and 211At. The choice of radionuclide to be incorporated into compounds of formula (I) will depend on the specific analytical or pharmaceutical application. Therefore, for in vitro labeling of mGlu5 receptors and for competition assays compounds that incorporate 3H, 125I or 77Br would be preferred. For diagnostic and investigating imaging agents (PET or SPECT) compounds that incorporate a radionuclide selected from 11C, 18F, 123I or 76Br are preferred.

The agents of the invention are therefore useful, for instance, for determining the levels of receptor occupancy of a drug acting at mGluR5, or diagnostic purposes for diseases resulting from an imbalance or dysfunction of mGluR5, and for monitoring the effectiveness of pharmacotherapies of such diseases.

In accordance with the above, the present invention provides an agent of the invention for use as a marker for neuroimaging.

In a further aspect, the present invention provides a composition for labeling brain and peripheral nervous system structures involving mGlu5 receptors in vivo and in vitro comprising an agent of the invention.

In still a further aspect, the present invention provides a method for labeling brain and peripheral nervous system structures involving mGluR5 in vitro or in vivo, which comprises contacting brain tissue with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

A list of Abbreviations used is given below.

AcOH acetic acid
aq. aqueous
BOC tert-butoxycarbonyl
n-BuLi n-butyl lithium
d day(s)
DCM dichloromethane
DMF N,N′-dimethylformamide
DMSO dimethyl sulfoxide
EDC 1-ethyl-3-[3-(dimethylamino)propyl]-carbodiimide hydrochloride
EtOAc ethylacetate
EtOH ethanol
h hour(s)
HCl hydrochloric acid
Hex hexane
HOBt hydroxybenzotriazole
HPLC high pressure liquid chromatography
HV high vacuum
LC liquid chromatography
MeOH methanol
min minute(s)
Mp melting point
MS mass spectroscopy
MTBE methyl-tert.-butylether
org. organic
PrOH propanol
Rf retention factor (Thin Layer Chromatography)
rt room temperature
RT retention time (HPLC and UPLC)
TFA trifluoroacetic acid
THF tetrahydrofuran
TLC thin layer chromatography
UPLC ultra performance liquid chromatography

The following non-limiting examples illustrate the invention.

### EXAMPLE 1

### (4-Chloro-phenyl)-[5-(1-ethyl-1H-imidazol-2-yl)-pyridin-2-yl]-amine

A de-gassed solution of 2-bromo-1-ethyl-1H-imidazole (33.6 mg, 0.19 mmol), 6-(4-chloro-phenylamino)-pyridine-3-boronic acid (39.7 mg, 0.16 mmol) and Pd(PPh₃)₄ (18.5 mg, 0.02 mmol) in benzene (1 ml), MeOH (0.3 ml) and 2M aq Na₂CO₃ (0.4 ml) were treated for 40 min at 120° C. in a microwave oven. The solvents were evaporated under reduced pressure and the residue purified by preparative thin layer chromatography using EtOAc/EtOH/NH₄OH 9:1:0.1 as mobile phase. 13 mg (26%) of the desired product were isolated as an amorphous solid. MS (LC/MS): 299 [M+H]. TLC Rf: 0.39 (EtOAc/EtOH/NH₄OH 9:1:0.1).

The starting materials were prepared as described hereafter:

### (5-Bromo-pyridin-2-yl)-(4-chloro-phenyl)-amine

2,5-Dibromo-pyridine (5.31 g) and 4-chloro-phenylamine (5.72 g) were mixed and heated to 170° C. for 3 h. The mixture was cooled and added to a 1M aqueous solution of Na₂CO₃. Extraction with Et₂O (2×), drying of the combined organic extracts, evaporation and crystallization from Et₂O/hexane afforded the desired product (3.85 g, 61%) as slightly purple crystals. M.p. 112-116° C.

### 6-(4-Chloro-phenylamino)-pyridine-3-boronic acid

A solution of (5-bromo-pyridin-2-yl)-(4-chloro-phenyl)-amine (992 mg, 3.5 mmol) in THF (28 ml) was cooled to −70° C. and then treated with a solution of n-BuLi in hexanes (1.6 M, 5.47 ml, 8.75 mmol) during 40 min. After stirring the mixture for additional 10 min at −70° C., triisopropylborate (1.01 ml, 4.2 mmol) was added during 15 min, and the mixture allowed to warm up to rt during 3.5 h. Water (5.5 ml) was added dropwise and THF evaporated under reduced pressure. The aqueous residue was diluted with water and extracted with Et₂O. The organic extracts were washed with water, all aqueous phases combined and neutralized with 2M HCl. The precipitation is collected by filtration and dried to afford the desired boronic acid (275 mg, 32%). MS (LC/MS): 249 [M+H].

### 2-Bromo-1-ethyl-1H-imidazole

A solution of 1-ethyl-1H-imidazole (0.91 g, 9.5 mmol) in acetonitrile (20 ml) was treated with BrCN (2.5 M in acetonitrile, 4 ml, 10 mmol) and the mixture stirred at room temperature for 4d. The solvent was evaporated under reduced pressure, water added to the residue and the mixture extracted with EtOAc. Drying of the organic extracts with Na₂SO₄ and evaporation leads to the crude product (0.9 g, 54%) which is used for the next step without further purification.

Following the same procedure, the following compounds can be obtained:

### EXAMPLE 2

### (4-Chloro-phenyl)-[5-(1-methyl-1H-imidazol-2-yl)-pyridin-2-yl]-amine

MS (LC/MS): 285 [M+H]

TLC Rf: 0.07 (EtOAc)

### EXAMPLE 3

### (4-Chloro-phenyl)-[5-(1-propyl-1H-imidazol-2-yl)-pyridin-2-yl]-amine

MS (LC/MS): 313 [M+H]

TLC Rf: 0.14 (EtOAc)

### EXAMPLE 4

### (4-Chloro-phenyl)-[5-(1-isopropyl-1H-imidazol-2-yl)-pyridin-2-yl]-amine

MS (LC/MS): 313 [M+H]

TLC Rf: 0.45 (EtOAc/EtOH/NH₄OH 9:1:0.1)

### EXAMPLE 5

### (4-Chloro-phenyl)-[5-(1-isobutyl-1H-imidazol-2-yl)-pyridin-2-yl]-amine

MS (LC/MS): 327 [M+H]

TLC Rf: 0.45 (EtOAc/EtOH/NH₄OH 9:1:0.1)

### EXAMPLE 6

### (4-Chloro-phenyl)-[5-(1-cyclopropylmethyl-1H-imidazol-2-yl)-pyridin-2-yl]-amine

MS (LC/MS): 325 [M+H]

TLC Rf: 0.15 (EtOAc)

### EXAMPLE 7

### (4-Chloro-phenyl)-[5-(1-yclohexyl-1H-imidazol-2-yl)-pyridin-2-yl]-amine

MS (LC/MS): 353 [M+H]

TLC Rf: 0.15 (EtOAc/EtOH/NH₄OH 9:1:0.1)

### EXAMPLE 8

### [5-(1-Benzyl-1H-imidazol-2-yl)-pyridin-2-yl]-(4-chloro-phenyl)-amine

MS (LC/MS): 361 [M+H]

TLC Rf: 0.18 (EtOAc)

### EXAMPLE 9

### (4-Chloro-phenyl)-[5-(1-phenyl-1H-imidazol-2-yl)-pyridin-2-yl]-amine

MS (LC/MS): 347 [M+H]

TLC Rf: 0.15 (EtOAc)

### EXAMPLE 10

### (4-Chloro-phenyl)-[5-(3-isopropyl-3H-imidazol-4-yl)-pyridin-2-yl]-amine

MS (LC/MS): 313 [M+H]

TLC Rf: 0.35 (EtOAc/EtOH/NH₄OH 9:1:0.1)

### EXAMPLE 11

### (4-Chloro-phenyl)-[5-(1-isopropyl-1H-imidazol-4-yl)-pyridin-2-yl]-amine

MS (LC/MS): 313 [M+H]

TLC Rf: 0.28 (EtOAc/EtOH/NH₄OH 9:1:0.1)

### EXAMPLE 12

### (4-Chloro-phenyl)-[5-(4-isopropyl-4H-[1,2,4]triazol-3-yl)-pyridin-2-yl]-amine

MS (LC/MS): 314 [M+H]

TLC Rf: 0.16 (EtOAc/EtOH/NH₄OH 9:1:0.1)

### EXAMPLE 13

### (4-Chloro-phenyl)-[5-(5,6,7,8-tetrahydro-[1,2,4]triazolo[4,3-a]pyridin-3-yl)-pyridin-2-yl]-amine

MS (LC/MS): 326 [M+H]

TLC Rf: 0.06 (EtOAc/EtOH/NH₄OH 9:1:0.1)

### EXAMPLE 14

### (4-Chloro-phenyl)-(5-[1,2,4]triazolo[4,3-a]pyridin-3-yl-pyridin-2-yl)-amine

MS (LC/MS): 313 [M+H]

### EXAMPLE 15

### [3-Chloro-5-(1-ethyl-1H-imidazol-2-yl)-pyridin-2-yl]-(4-chloro-phenyl)-amine

MS (LC/MS): 333 [M+H]

TLC Rf: 0.39 (EtOAc)

### EXAMPLE 16

### (3-Chloro-5-imidazo[1,5-a]pyridin-3-yl-pyridin-2-yl)-(4-chloro-phenyl)-amine

MS (LC/MS): 357 [M+H]

TLC Rf: 0.68 (DCM/MeOH 9:1)

### EXAMPLE 17

### (4-Chloro-phenyl)-[3-chloro-5-(5,6,7,8-tetrahydro-imidazo[1,5-a]pyridin-3-yl)-pyridin-2-yl]-amine

MS (LC/MS): 360 [M+H]

TLC Rf: 0.51 (DCM/MeOH 9:1)

### EXAMPLE 18

### [3-Chloro-5-(1-ethyl-1H-imidazol-2-yl)-pyridin-2-yl]-(6-methyl-pyridin-3-yl)-amine

MS (LC/MS): 314 [M+H]

TLC Rf: 0.34 (DCM/MeOH 9:1)

### EXAMPLE 19

### (4-Chloro-phenyl)-[3-chloro-5-(1-propyl-1H-imidazol-2-yl)-pyridin-2-yl]-amine

MS (LC/MS): 348 [M+H]

TLC Rf: 0.48 (DCM/MeOH 9:1)

### EXAMPLE 20

### [3-Chloro-5-(1-ethyl-4,5-dimethyl-1H-imidazol-2-yl)-pyridin-2-yl]-(4-chloro-phenyl)-amine

MS (LC/MS): 362 [M+H]

TLC Rf: 0.26 (DCM/MeOH 95:5)

### EXAMPLE 21

### (4-Chloro-phenyl)-[3-chloro-5-(1H-tetrazol-5-yl)-pyridin-2-yl]-amine

A solution of 5-chloro-6-(4-chloro-phenylamino)-nicotinonitrile (1.0 g, 3.71 mmol) and tributyltin azide (2.85 ml, 10.6 mmol) was heated to 100° C. for 11 h, and the solvent was then evaporated in vacuo. Purification by flash chromatography (DCM/MeOH 100:0 to 80:20) and crystallization from EtOAc gave the desired product as beige crystals (0.60 g, 53%). UPLC (5-100% CH₃CN): RT=1.379 min, MS (ES+): 307 [M⁺].

The starting materials were prepared as described below

### 6-Amino-5-chloro-nicotinonitrile

A solution of 6-amino-nicotinonitrile (1.0 g, 8.2 mmol) in DMF (10 ml) was treated with N-chlorosuccinimide (1.26 g, 9.1 mmol) and the mixture was heated to 80° C. for 4 h. It was then allowed to cool to rt. The mixture was then poured onto ice/water and the precipitate was filtered. The filter cake was washed with water and then dried in HV to give pure 6-amino-5-chloro-nicotinonitrile (1.1 g, 87%). UPLC (5-100% CH₃CN): RT=0.790 min.

### 5,6-Dichloro-nicotinonitrile

CuCl₂ (5.36 g, 15.9 mmol) and tert-butyl nitrite (2.53 ml, 19.2 mmol) were added in succession to a flask containing CH₃CN (100 ml) and the mixture was heated to 65° C. A solution of 6-amino-5-chloro-nicotinonitrile (2.0 g, 12.8 mmol) in CH₃CN (1 ml) was then added dropwise and the formation of gas was observed. The temperature was kept at 65° C. for 4 h and the mixture was then cooled and added to a 2N aq. solution of HCl. Extraction with EtOAc, drying over Na₂SO₄, evaporation and purification by flash chromatography (Hex/EtOAc 100:0 to 80:20) provided 5,6-dichloro-nicotinonitrile (1.40 g, 63%). UPLC (5-100% CH₃CN): RT=1.120 min.

### 5-Chloro-6-(4-chloro-phenylamino)-nicotinonitrile

A de-gassed solution of [Pd(OAc)₂] (58.0 mg, 0.24 mmol) and rac-BINAP (162 mg, 0.26 mmol) in toluene (50 ml) was stirred for 10 min at rt, and 4-chloroaniline (1.53 g, 11.9 mmol) and 5,6-dichloro-nicotinonitrile (1.40 g, 7.93 mmol) were then added. The mixture was stirred at rt for another 10 min, treated with K₂CO₃ (5.54 g, 39.7 mmol) and heated to 100° C. for 16 h. The solvent was then evaporated in vacuo and the crude product was purified by flash chromatography (Hex/DCM 100:0 to 0:100) to afford 5-chloro-6-(4-chloro-phenylamino)-nicotinonitrile (1.48 g, 71%). UPLC (5-100% CH₃CN): RT=1.635 min.

### EXAMPLE 22

### (4-Chloro-phenyl)-[3-chloro-5-(1-propyl-1H-tetrazol-5-yl)-pyridin-2-yl]-amine

A solution of (4-chloro-phenyl)-[3-chloro-5-(1H-tetrazol-5-yl)-pyridin-2-yl]-amine (120 mg, 0.39 mmol) in DMF (4 ml) was treated with NaH (10.4 mg, 0.41 mmol). The mixture was stirred for 20 min at rt and 1-iodopropane (87 μl, 0.75 mmol) was the added. After 30 min, the mixture was diluted with water and extracted with EtOAc. The combined org. phases were dried over Na₂SO₄ and concentrated in vacuo. Purification by flash chromatography (Hex/EtOAc 100:0 to 50:50) furnished 4-chloro-phenyl)-[3-chloro-5-(1-propyl-1H-tetrazol-5-yl)-pyridin-2-yl]-amine (60 mg, 44%). UPLC (5-100% CH₃CN): RT=1.924 min, MS (ES+): 349 [M⁺].

Following the same procedure, the following compound can be obtained:

### EXAMPLE 23

### [3-Chloro-5-(1-isobutyl-1H-tetrazol-5-yl)-pyridin-2-yl]-(4-chloro-phenyl)-amine

MS (ES+): 363 [M⁺]

UPLC (5-100% CH₃CN): RT=2.022 min

### EXAMPLE 24

### (4-Chloro-phenyl)-[3-chloro-5-(5,6,7,8-tetrahydro-[1,2,4]triazolo[4,3-a]pyridin-3-yl)-pyridin-2-yl]-amine

A solution of 5-chloro-6-(4-chloro-phenylamino)-nicotinic acid hydrazide (200 mg, 0.67 mmol) and 6-methoxy-2,3,4,5-tetrahydro-pyridine (76.2 mg, 0.67 mmol) in EtOH (15 ml) was heated to reflux for 20 h. The mixture was the cooled to rt and concentrated in vacuo. The crude product was purified by flash chromatography (DCM/MeOH 100:0 to 90:10) to afford the desired product as a white solid (240 mg, 99%). UPLC (5-100% CH₃CN): RT=1.190 min, MS (ES+): 360 [M⁺].

The starting materials were prepared as described below

### 5,6-Dichloro-nicotinic acid methyl ester

A solution of 5,6-dichloro-nicotinic acid (10.0 g, 51.0 mmol) and DMF (7 μl) in SOCl₂ (49.5 ml) was heated to 105° C. for 1 h. The mixture was then concentrated in vacuo and treated with cooled MeOH (10 ml, 0° C.). The solution was allowed to warm slowly to rt over 30 min. The solvent was then evaporated in vacuo and the crude product was purified by flash chromatography (Hex/EtOAc 1:1) to provide 5,6-dichloro-nicotinic acid methyl ester (10.3 g, 99%). UPLC (5-100% CH₃CN): RT=1.374 min.

### 5-Chloro-6-(4-chloro-phenylamino)-nicotinic acid methyl ester

A solution of [Pd(OAc)₂] (365 mg, 1.59 mmol) and rac-BINAP (1.02 g, 1.61 mmol) in de-gassed toluene (20 ml) was treated with a solution of 5,6-dichloro-nicotinic acid methyl ester (10.3 g, 50.0 mmol) in de-gassed toluene (10 ml) and a solution of 4-chloroaniline (9.66 g, 75.0 mmol) in de-gassed toluene (10 ml). The mixture was stirred at rt for 15 min and K₂CO₃ (34.9 g, 250 mmol) was added. The suspension was heated to reflux for 16 h, and the solvent was then evaporated in vacuo. The residue was taken up in DCM, acidified with 1N aq. HCl, and extracted with DCM. The combined organic layers were dried over Na₂SO₄, and concentrated in vacuo. Purification by flash chromatography (Hex/EtOAc 100:0 to 80:20) and crystallization in i-PrOH gave 5-chloro-6-(4-chloro-phenylamino)-nicotinic acid methyl ester (5.69 g, 38%). UPLC (5-100% CH₃CN): RT=1.755 min.

### 5-Chloro-6-(4-chloro-phenylamino)-nicotinic acid hydrazide

A mixture of 5-chloro-6-(4-chloro-phenylamino)-nicotinic acid methyl ester (4.6 g, 15.5 mmol) and hydrazine monohydrate (61.4 ml, 1.24 mol) in EtOH (20 ml) was heated to reflux for 1 h, then cooled to rt and diluted with water (20 ml) and EtOAc (20 ml). After separation of the organic phase, the aq. layer was extracted with EtOAc. The combined org. layers were washed with brine, dried over Na₂SO₄, and concentrated in vacuo to give crude 5-chloro-6-(4-chloro-phenylamino)-nicotinic acid hydrazide (4.55 g, 99%), which was used in the next step without further purification. UPLC (5-100% CH₃CN): RT=1.040 min.

Following the same procedures, the following compound can be obtained:

### EXAMPLE 25

### (4-Chloro-phenyl)-[3-chloro-5-(6,7,8,9-tetrahydro-5H-[1,2,4]triazolo[4,3-a]azepin-3-yl)-pyridin-2-yl]-amine

MS (ES+): 374 [M⁺]

UPLC (5-100% CH₃CN): RT=1.253 min

### EXAMPLE 26

### [3-Chloro-5-(5,6,7,8,9,10-hexahydro-[1,2,4]triazolo[4,3-a]azocin-3-yl)-pyridin-2-yl]-(4-chloro-phenyl)-amine

MS (LC/MS): 388 [M⁺]

UPLC (5-100% CH₃CN): RT=1.299 min

### EXAMPLE 27

### [3-Chloro-5-(6,7,8,9,10,11-hexahydro-5H-[1,2,4]triazolo[4,3-a]azonin-3-yl)-pyridin-2-yl]-(4-chloro-phenyl)-amine

MS (LC/MS): 402 [M⁺]

UPLC (5-100% CH₃CN): RT=1.360 min

### EXAMPLE 28

### [3-Chloro-5-(1-ethyl-1H-pyrrol-2-yl)-pyridin-2-yl]-(4-chloro-phenyl)-amine

A solution of (4-chloro-phenyl)-[3-chloro-5-(1H-pyrrol-2-yl)-pyridin-2-yl]-amine (60.0 mg, 0.20 mmol) in DMF (4 ml) was treated with NaH (5.3 mg, 0.21 mmol), stirred at rt for 30 min, and 1-iodoethane (32 μl, 0.39 mmol) was then added. The mixture was stirred for 16 h at rt, then diluted with water and extracted with EtOAc. The combined org. phases were concentrated in vacuo and purified by flash chromatography (Hex/EtOAc 100:0 to 30:70) and preparative HPLC(CH₃CN 5 to 100%) to provide the desired product (6.4 mg, 10%). UPLC (5-100% CH₃CN): RT=1.961 min, MS (ES+): 332 [M⁺].

The starting materials were prepared as described below:

### (5-Bromo-3-chloro-pyridin-2-yl)-(4-chloro-phenyl)-amine

A solution of 5-bromo-2,3-dichloropyridine (10.0 g, 43.2 mmol) in anhydrous THF (200 ml) was treated portionwise with NaH (2.13 g, 84 mmol) at rt. After 1 h a solution of 4-chloroaniline (11.1 g, 86.1 mmol) in THF (100 ml) was added dropwise and the suspension was then heated to reflux for 14 h. The mixture was then allowed to cool to rt and the reaction was quenched by adding sat. aq. solution of Na₂CO₃. The solvent was evaporated in vacuo and the aq. layer was extracted with EtOAc. The combined org. phases were dried over Na₂SO₄, concentrated in vacuo and the crude product was purified by flash chromatography (Hex/EtOAc 100:0 to 80:20) to give (5-bromo-3-chloro-pyridin-2-yl)-(4-chloro-phenyl)-amine (9.3 g, 68%). UPLC (5-100% CH₃CN): RT=1.989 min.

### (4-Chloro-phenyl)-[3-chloro-5-(1H-pyrrol-2-yl)-pyridin-2-yl]-amine

A suspension of (5-bromo-3-chloro-pyridin-2-yl)-(4-chloro-phenyl)-amine (900 mg, 2.83 mmol), N-(t-butoxycarbonyl)pyrrole-2-boronic acid (616 mg, 2.83 mmol), Na₂CO₃ (455 mg, 4.25 mmol) and [Pd(PPh₃)₄] (169 mg, 0.14 mmol) in toluene/EtOH/water (5:5:1, 5 ml) was heated for 4 h at 120° C. in the microwave oven. The mixture was then concentrated in vacuo and the crude product was purified by flash chromatography (Hex/EtOAc 100:0 to 50:50) and preparative HPLC(CH₃CN 5 to 100%) to afford (4-chloro-phenyl)-[3-chloro-5-(1H-pyrrol-2-yl)-pyridin-2-yl]-amine (80 mg, 9%). UPLC (5-100% CH₃CN): RT=1.696 min.

### EXAMPLE 29

### [3-Chloro-5-(2,5-dimethyl-2H-pyrazol-3-yl)-pyridin-2-yl]-(4-chloro-phenyl)-amine

Methyl hydrazine (49.1 mg, 1.04 mmol) in MeOH (0.3 ml) was acidified with HCl in i-PrOH to pH 1-2 and the mixture was stirred at rt for 30 min. The solvent was then evaporated in vacuo and solid obtained was added to a solution of 1-[5-chloro-6-(4-chloro-phenylamino)-pyridin-3-yl]-butane-1,3-dione (150 mg, 0.46 mmol) in EtOH (15 ml). The mixture was heated to 90° C. overnight, cooled to rt and concentrated in vacuo. The residue was taken up in water and extracted with EtOAc. The combined org. layers were washed with brine, dried over Na₂SO₄, concentrated in vacuo, and the crude product was purified by flash chromatography (Hex/EtOAc 100:0 to 50:50) and preparative TLC (Hex/EtOAc 1:1) to provide the desired product as a brown solid (65.2 mg, 42%). UPLC (5-100% CH₃CN): RT=1.579 min, MS (ES+): 333 [M⁺].

The starting materials were prepared as described below:

### 1-[5-Chloro-6-(4-chloro-phenylamino)-pyridin-3-yl]-ethanone

A solution of (5-bromo-3-chloro-pyridin-2-yl)-(4-chloro-phenyl)-amine (2.0 g, 6.29 mmol), tributyl(1-ethoxyvinyl)stannane (2.95 g, 8.18 mmol), [Pd(PPh₃)₄] (362 mg, 0.31 mmol) and triethylamine (1.31 ml, 9.4 mmol) in de-gassed dioxane was heated to reflux for 24 h. The solvent was then evaporated in vacuo and the residue was filtered through a thick pad of SiO₂. The solid obtained was then taken up in anhydrous THF (100 ml), cooled to 0° C., and Treated with an 1N aq. solution of HCl. The solution was stirred for 2 h at rt and then neutralized with sat. aq. NaHCO₃. This mixture was extracted with EtOAc and the combined org. phases were washed with brine, dried over Na₂SO₄, and concentrated in vacuo. Purification by flash chromatography (Hex/EtOAc 100:0 to 80:20) and crystallization from hexane gave 1-[5-chloro-6-(4-chloro-phenylamino)-pyridin-3-yl]-ethanone (1.07 g, 73%). UPLC (5-100% CH₃CN): RT=1.602 min.

### 1-[5-Chloro-6-(4-chloro-phenylamino)-pyridin-3-yl]-butane-1,3-dione

A solution of LHMDS (1M, 1.4 ml, 1.4 mmol) in anhydrous THF (4 ml) was cooled to −12° C. and then treated with a solution of 1-[5-chloro-6-(4-chloro-phenylamino)-pyridin-3-yl]-ethanone (200 mg, 0.71 mmol) in anhydrous THF (2 ml). The mixture was stirred for 30 min at this temperature and dry EtOAc (0.28 ml, 2.85 mmol) was then added. The solution was kept below −10° C. for 1 h and was then allowed to warm to rt overnight. The mixture was then diluted with water and the pH was adjusted to 6 with 2N aq. HCl. It was then extracted with EtOAc, and the combined org layers were washed with brine, dried and concentrated in vacuo to give crude 1-[5-chloro-6-(4-chloro-phenylamino)-pyridin-3-yl]-butane-1,3-dione (215 mg, 65%) which was used as it is in the next reaction. UPLC (5-100% CH₃CN): RT=1.881 min.

### EXAMPLE 30

### [3-Chloro-5-(1,4-dimethyl-1H-imidazol-2-yl)-pyridin-2-yl]-(4-chloro-phenyl)-amine

A suspension of [3-chloro-5-(4-methyl-1H-imidazol-2-yl)-pyridin-2-yl]-(4-chloro-phenyl)-amine (150 mg, 0.47 mmol), iodomethane (22 μl, 0.34 mmol) and K₂CO₃ (96 mg, 0.69 mmol) in dry DMF (2 ml) was stirred at rt for 16 h. The mixture was then poured onto water and extracted with EtOAc. The combined org phases were dried over Na₂SO₄, concentrated in vacuo and purified by flash chromatography (Hex/EtOAc 100:0 to 20:80) to furnish the desired product (45 mg, 29%). UPLC (5-100% CH₃CN): RT=1.133 min, MS (ES+): 333 [M⁺].

### EXAMPLE 31

### [3-Chloro-5-(1,5-dimethyl-1H-imidazol-2-yl)-pyridin-2-yl]-(4-chloro-phenyl)-amine

During the purification of crude [3-chloro-5-(1,4-dimethyl-1H-imidazol-2-yl)-pyridin-2-yl]-(4-chloro-phenyl)-amine (Example 30), another regioisomer, [3-chloro-5-(1,5-dimethyl-1H-imidazol-2-yl)-pyridin-2-yl]-(4-chloro-phenyl)-amine, could be isolated by preparative TLC (Hex/EtOAc 1:1) as a white solid (16 mg, 10%). UPLC (5-100% CH₃CN): RT=1.136 min, MS (ES+): 333 [M⁺].

The starting materials were prepared as described below:

### 5-Chloro-6-(4-chloro-phenylamino)-nicotinamidine

A solution of 5-chloro-6-(4-chloro-phenylamino)-nicotinonitrile (800 mg, 3.03 mmol) and NaOMe (253 mg, 4.54 mmol) in MeOH (20 ml) was stirred for 16 h at rt. NH₄Cl (180 mg, 3.33 mmol) was then added and the mixture was heated to 65° C. for 2 h. The solvent was evaporated and the residue was taken up in EtOH and stirred for 2 h at rt. The precipitate was filtered to give 5-chloro-6-(4-chloro-phenylamino)-nicotinamidine (520 mg, 61%). UPLC (5-100% CH₃CN): RT=1.020 min.

In some cases, an excess of NH₄Cl had was used in order to push the reaction to completion. The excess of NH₄Cl could not always be separated from the 5-chloro-6-(4-chloro-phenylamino)-nicotinamidine, but NH₄Cl did not have any negative influence on the next cyclization step (see examples 34 and 37).

### [3-Chloro-5-(4-methyl-1H-imidazol-2-yl)-pyridin-2-yl]-(4-chloro-phenyl)-amine

A suspension of 5-chloro-6-(4-chloro-phenylamino)-nicotinamidine (500 mg, 1.78 mmol), chloroacetone (115 μl, 1.30 mmol), and NH₄Cl (140 mg, 2.59 mmol) in NH₄OH (4 ml) was heated to 80° C. for 5 h. It was then allowed to cool to rt and then diluted with water. The mixture was extracted with EtOAc, and the combined org. layers were dried over Na₂SO₄ and concentrated in vacuo. Purification by flash chromatography (Hex/EtOAc 100:0 to 0:100) and crystallization from hexane afforded [3-chloro-5-(4-methyl-1H-imidazol-2-yl)-pyridin-2-yl]-(4-chloro-phenyl)-amine (205 mg, 36%). UPLC (5-100% CH₃CN): RT=1.108 min.

Following the same procedures, the following compounds can be obtained:

### EXAMPLE 32

### [3-Chloro-5-(1-ethyl-4-methyl-1H-imidazol-2-yl)-pyridin-2-yl]-(4-chloro-phenyl)-amine

MS (ES+): 347 [M⁺]

UPLC (5-100% CH₃CN): RT=1.202 min

### EXAMPLE 33

### [3-Chloro-5-(4-methyl-1-propyl-1H-imidazol-2-yl)-pyridin-2-yl]-(4-chloro-phenyl)-amine

MS (ES+): 361 [M⁺]

UPLC (5-100% CH₃CN): RT=1.281 min

### EXAMPLE 34

### [3-Chloro-5-(4-ethyl-1-propyl-1H-imidazol-2-yl)-pyridin-2-yl]-(4-chloro-phenyl)-amine

A solution of [3-chloro-5-(4-ethyl-1H-imidazol-2-yl)-pyridin-2-yl]-(4-chloro-phenyl)-amine (100 mg, 0.30 mmol) in DMF (4 ml) was treated with NaH (8.0 mg, 0.32 mmol) and the mixture was stirred for 30 min at rt. 1-iodopropane (69 μl, 0.60 mmol) was added and the mixture was stirred for 4 h at rt and then 1 h at 60° C. The mixture was then diluted with water and extracted with EtOAc. The combined org. layers were dried, concentrated in vacuo and the crude product was purified by flash chromatography (Hex/EtOAc 100:0 to 40:60) to give the desired product (40 mg, 36%). UPLC (5-100% CH₃CN): RT=1.334 min, MS (ES+): 375 [M⁺].

The starting materials were prepared as described below:

### [3-Chloro-5-(4-ethyl-1H-imidazol-2-yl)-pyridin-2-yl]-(4-chloro-phenyl)-amine

A suspension of 5-chloro-6-(4-chloro-phenylamino)-nicotinamidine (37% pure, 1.5 g, 1.97 mmol), 1-bromo-2-butanone (255 μl, 2.37 mmol), and KHCO₃ (2.0 g, 19.8 mmol) in anhydrous THF (40 ml) was heated to 80° C. and then maintained at 60° C. for 2 h. The mixture was then diluted with water and extracted with EtOAc. The combined org. phases were dried and concentrated in vacuo. Crystallization from EtOAc/Hex gave [3-chloro-5-(4-ethyl-1H-imidazol-2-yl)-pyridin-2-yl]-(4-chloro-phenyl)-amine (640 mg, 97%). UPLC (5-100% CH₃CN): RT=1.157 min.

Following the same procedures, the following compounds can be obtained:

### EXAMPLE 35

### [5-(1-Butyl-4-ethyl-1H-imidazol-2-yl)-3-chloro-pyridin-2-yl]-(4-chloro-phenyl)-amine

MS (ES+): 389 [M⁺]

UPLC (5-100% CH₃CN): RT=1.405 min

### EXAMPLE 36

### [3-Chloro-5-(1,4-diethyl-1H-imidazol-2-yl)-pyridin-2-yl]-(4-chloro-phenyl)-amine

MS (ES+): 361 [M⁺]

UPLC (5-100% CH₃CN): RT=1.257 min

### EXAMPLE 37

### [5-(5-tert-Butyl-1H-imidazol-2-yl)-3-chloro-pyridin-2-yl]-(4-chloro-phenyl)-amine

A solution of 5-chloro-6-(4-chloro-phenylamino)-nicotinamidine (37% pure, 1.0 g, 1.32 mmol), 1-chloro-3,3-dimethyl-2-butanone (252 μl, 2.63 mmol), and KHCO₃ (1.33 g, 13.2 mmol) in anhydrous THF (40 ml) was heated to 80° C. for 5 h. The mixture was then diluted with water and extracted with EtOAc. The combined org. phases were dried and concentrated in vacuo. Purification by flash chromatography (Hex/EtOAc 100:0 to 50:50) provided the desired product (385 mg, 81%). UPLC (5-100% CH₃CN): RT=1.253 min, MS (ES+): 361 [M⁺].

### EXAMPLE 38

### [5-(4-tert-Butyl-1-methyl-1H-imidazol-2-yl)-3-chloro-pyridin-2-yl]-(4-chloro-phenyl)-amine

A solution of [5-(5-tert-butyl-1H-imidazol-2-yl)-3-chloro-pyridin-2-yl]-(4-chloro-phenyl)-amine (100 mg, 0.28 mmol) in anhydrous DMF (4 ml) was treated with NaH (7.3 mg, 0.29 mmol) and the mixture was stirred for 30 min at rt. Iodomethane (35 μl, 0.55 mmol) was then added and the solution was stirred for 16 h at rt. The mixture was diluted with water and extracted with EtOAc. The combined org. layers were dried over Na₂SO₄, concentrated in vacuo and purified by flash chromatography (Hex/EtOAc 100:0 to 50:50) and preparative TLC (DCM/MeOH 9:1) to provide [5-(4-tert-butyl-1-methyl-1H-imidazol-2-yl)-3-chloro-pyridin-2-yl]-(4-chloro-phenyl)-amine (9 mg, 9%). UPLC (5-100% CH₃CN): RT=1.284 min, MS (ES+): 375 [M⁺].

Following the same procedures, the following compounds can be obtained:

### EXAMPLE 39

### [5-(4-tert-Butyl-1-ethyl-1H-imidazol-2-yl)-3-chloro-pyridin-2-yl]-(4-chloro-phenyl)-amine

MS (ES+): 389 [M⁺]

UPLC (5-100% CH₃CN): RT=1.356 min

### EXAMPLE 40

### [5-(4-tert-Butyl-1-propyl-1H-imidazol-2-yl)-3-chloro-pyridin-2-yl]-(4-chloro-phenyl)-amine

MS (ES+): 403 [M⁺]

UPLC (5-100% CH₃CN): RT=1.425 min

### EXAMPLE 41

### [5-(1-Butyl-4-tert-butyl-1H-imidazol-2-yl)-3-chloro-pyridin-2-yl]-(4-chloro-phenyl)-amine

MS (ES+): 417 [M⁺]

UPLC (5-100% CH₃CN): RT=1.495 min

### EXAMPLE 42

### [3-Chloro-5-(4,5-dimethyl-1-propyl-1H-imidazol-2-yl)-pyridin-2-yl]-(4-chloro-phenyl)-amine

A solution of [3-chloro-5-(4,5-dimethyl-1H-imidazol-2-yl)-pyridin-2-yl]-(4-chloro-phenyl)-amine (70 mg, 0.21 mmol) in anhydrous DMF (4 ml) was treated with NaH (5.6 mg, 0.22 mmol) and the mixture was stirred for 30 min at rt. 1-iodopropane (49 μl, 0.42 mmol) was added and the mixture was stirred for 16 h at rt. It was then poured onto water and extracted with EtOAc. The combined org. layers were dried over Na₂SO₄ and concentrated in vacuo. Purification by flash chromatography (Hex/EtOAc 100:0 to 50:50) and preparative HPLC (CH₃CN 5 to 100%) furnished the desired product (6 mg, 8%). UPLC (5-100% CH₃CN): RT=1.320 min, MS (ES+): 375 [M⁺].

The starting materials were prepared as described below:

### [3-Chloro-5-(4,5-dimethyl-1H-imidazol-2-yl)-pyridin-2-yl]-(4-chloro-phenyl)-amine

A solution of 5-chloro-6-(4-chloro-phenylamino)-nicotinamidine (37% pure, 1.5 g, 1.97 mmol) and 3-chloro-2-butanone (822 μl, 7.90 mmol) in NH₄OH (26% NH₃ in water, 150 ml) was heated to reflux for 16 h. The mixture was then cooled to rt and the precipitate was filtered, washed with water. Purification by flash chromatography (Hex/EtOAc 100:0 to 0:100) and crystallization from EtOAc afforded [3-chloro-5-(4,5-dimethyl-1H-imidazol-2-yl)-pyridin-2-yl]-(4-chloro-phenyl)-amine (320 mg, 49%). UPLC (5-100% CH₃CN): RT=1.161 min.

### EXAMPLE 43

### 2-[5-Chloro-6-(4-chloro-phenylamino)-pyridin-3-yl]-1,3,5-triethyl-4-methyl-3H-imidazol-1-ium iodide

A solution of [3-chloro-5-(5-ethyl-4-methyl-1H-imidazol-2-yl)-pyridin-2-yl]-(4-chloro-phenyl)-amine (100 mg, 0.29 mmol) in anhydrous DMF (4 ml) was treated with NaH (7.7 mg, 0.30 mmol) and the mixture was stirred for 30 min at rt. Iodoethane (26 μl, 0.32 mmol) was added and the mixture was stirred for 4 h at rt. The mixture was then heated to 60° C. for 16 h and then concentrated in vacuo. The crude product was purified by flash chromatography (DCM/MeOH 100:0 to 90:10) to provide the desired product (10 mg, 8%). UPLC (5-100% CH₃CN): RT=1.397 min, MS (ES+): 404 [M⁺-I].

The starting materials were prepared as described below:

### [5-Chloro-6-(4-chloro-phenylamino)-pyridin-3-yl]-methanol

A suspension of [Pd(OAc)₂] (201 mg, 0.88 mmol) and rac-BINAP (561 mg, 0.88 mmol) in de-gassed toluene (200 ml) was stirred for 10 min at rt, prior to adding (5,6-dichloropyridin-3-yl)-methanol (5.0 g, 27.5 mmol) and 4-chloroaniline (5.32 g, 41.3 mmol). The mixture was stirred for another 10 min at rt and K₂CO₃ (19.2 g, 138 mmol) was then added. The mixture was heated to 120° C. for 4 h and the solvent was then evaporated. Purification by flash chromatography (Hex/EtOAc 100:0 to 0:100) gave [5-chloro-6-(4-chloro-phenylamino)-pyridin-3-yl]-methanol (3.4 g, 46%). UPLC (5-100% CH₃CN): RT=1.146 min.

### 5-Chloro-6-(4-chloro-phenylamino)-pyridine-3-carbaldehyde

A solution of [5-chloro-6-(4-chloro-phenylamino)-pyridin-3-yl]-methanol (3.0 g, 10.9 mmol) in DCM (200 ml) was treated with pyridinium chlorochromate (4.81 g, 21.9 mmol) and the mixture was stirred for 30 min at rt. The mixture was then diluted with EtOAc, and the precipitate was filtered. The filtrate was concentrated in vacuo and purified by flash chromatography (Hex/EtOAc 100:0 to 30:70) furnishing 5-chloro-6-(4-chloro-phenylamino)-pyridine-3-carbaldehyde (1.5 g, 51%). UPLC (5-100% CH₃CN): RT=1.564 min.

### [3-Chloro-5-(5-ethyl-4-methyl-1H-imidazol-2-yl)-pyridin-2-yl]-(4-chloro-phenyl)-amine

A mixture of 5-chloro-6-(4-chloro-phenylamino)-pyridine-3-carbaldehyde (1.5 g, 5.62 mmol), 2,3-pentanedione (447 μl, 4.15 mmol) and NH₄OAc (1.62 g, 20.8 mmol) in AcOH (15 ml) were heated to 180° C. for 2 h in a microwave oven. The mixture was then poured onto aq. NH₄OH solution and extracted with EtOAc. The combined org. phases were then dried and evaporated. Purification by flash chromatography (Hex/EtOAc 100:0 to 30:70) provided [3-chloro-5-(5-ethyl-4-methyl-1H-imidazol-2-yl)-pyridin-2-yl]-(4-chloro-phenyl)-amine (500 mg, 26%). UPLC (5-100% CH₃CN): RT=1.213 min.

### EXAMPLE 44

### [5-(5-Butyl-[1,2,3]triazol-1-yl)-3-chloro-pyridin-2-yl]-(4-chloro-phenyl)-amine

A solution of [5-(5-butyl-4-trimethylsilanyl-[1,2,3]triazol-1-yl)-3-chloro-pyridin-2-yl]-(4-chloro-phenyl)-amine (480 mg, 1.10 mmol) in anhydrous THF (10 ml) was treated with TBAF trihydrate (539 mg, 1.66 mmol) and heated to reflux for 18 h. The mixture was then cooled to rt, diluted with EtOAc, and washed with water. The org. phase was then dried over Na₂SO₄, filtered and concentrated in vacuo. Purification by flash chromatography (Hex/EtOAc 100:0 to 80:20) and crystallization from Hex/EtOAc gave the desired product (126 mg, 32%). LC (Zorbax, 50-100% CH₃CN): RT=2.808 min, LC/MS (ES+): 363 [M+H].

The starting materials were prepared as described below:

### (3-Chloro-5-nitro-pyridin-2-yl)-(4-chloro-phenyl)-amine

A suspension of NaH (2.07 g, 51.8 mmol) in anhydrous THF (60 ml) was treated with a solution of chloroaniline (6.68 g, 51.8 mmol) in THF (40 ml) and the mixture was stirred for 2 h at rt. A solution of 2,3-dichloro-5-nitro-pyridine (5.0 g, 25.9 mmol) in THF (40 ml) was then added and the mixture was heated to reflux for 18 h. It was then poured onto a sat. aq. solution of Na₂CO₃ and the THF was evaporated. The aq. phase was extracted with EtOAc and the combined org. layers were then dried and concentrated in vacuo. Purification by flash chromatography (Hex/EtOAc 9:1) and crystallization from Hex/EtOAc afforded (3-chloro-5-nitro-pyridin-2-yl)-(4-chloro-phenyl)-amine (2.36 g, 32%). LC/MS (ES+): 284, 286 [M+H].

### 3-Chloro-N-2-(4-chloro-phenyl)-pyridine-2,5-diamine

A solution of (3-chloro-5-nitro-pyridin-2-yl)-(4-chloro-phenyl)-amine (2.35 g, 8.27 mmol) in conc. HCl (20 ml) was treated portionwise with SnCl₂ dihydrate (5.71 g, 24.8 mmol) and the exothermic reaction was controlled with an ice/water bath. The mixture was then stirred for 18 h at rt, then cooled to 0° C. and rendered basic with 25% aq. NaOH solution. The mixture was then diluted with water and EtOAc and filtered. The filtrated was extracted with EtOAc and the combined org. layers were dried over Na₂SO₄, filtered and concentrated in vacuo. Purification by flash chromatography (Hex/EtOAc 100:0 to 75:25) and crystallization from Hex gave 3-chloro-N-2-(4-chloro-phenyl)-pyridine-2,5-diamine (1.7 g, 81%). LC/MS (ES+): 255, 257 [M+H].

### (5-Azido-3-chloro-pyridin-2-yl)-(4-chloro-phenyl)-amine

A solution of sodium azide (775 mg, 11.8 mmol) in tert-BuOH (6 ml) and water (1 ml) was treated with 3-chloro-N-2-(4-chloro-phenyl)-pyridine-2,5-diamine (1.0 g, 3.94 mmol) and tert-butyl nitrite (6.24 ml, 47.2 mmol). The mixture was the heated to 50° C. for 24 h and then diluted with EtOAc. It was then washed with water, dried over Na₂SO₄, filtered and concentrated in vacuo. Purification by flash chromatography (Hex/EtOAc 100:0 to 90:10) furnished (5-azido-3-chloro-pyridin-2-yl)-(4-chloro-phenyl)-amine (962 mg, 87%). LC/MS (ES+): 280, 282 [M+H].

### [5-(5-Butyl-4-trimethylsilanyl-[1,2,3]triazol-1-yl)-3-chloro-pyridin-2-yl]-(4-chloro-phenyl)-amine

A solution of (5-azido-3-chloro-pyridin-2-yl)-(4-chloro-phenyl)-amine (960 mg, 3.43 mmol) in toluene (15 ml) was treated with 1-trimethylsilyl-1-hexyne (769 μl, 3.77 mmol) and then heated to 50° C. for 4 d. The mixture was then diluted with EtOAc, washed with water, dried over Na₂SO₄, filtered and concentrated in vacuo. Purification by flash chromatography (Hex/EtOAc 100:0 to 90:10) provided [5-(5-butyl-4-trimethylsilanyl-[1,2,3]triazol-1-yl)-3-chloro-pyridin-2-yl]-(4-chloro-phenyl)-amine (490 mg, 33%). LC/MS (ES+): 435 [M+H].

Following the same procedures, the following compound can be obtained:

### EXAMPLE 45

### (4-Chloro-phenyl)-[3-chloro-5-(5-propyl-[1,2,3]triazol-1-yl)-pyridin-2-yl]-amine

LC/MS (ES+): 348, 350 [M⁺]

LC (Zorbax, 30-100% CH₃CN): RT=3.511 min

### EXAMPLE 46

### (4-Chloro-phenyl)-[3-chloro-5-(5-propyl-3H-[1,2,3]triazol-4-yl)-pyridin-2-yl]-amine

A solution of (3-chloro-5-pent-1-ynyl-pyridin-2-yl)-(4-chloro-phenyl)-amine (550 mg, 1.80 mmol) and sodium azide (592 mg, 9.02 mmol) in DMSO (10 ml) was heated to 150° C. for 5 d. The mixture was then allowed to cool to rt, diluted with EtOAc, washed with water, dried over Na₂SO₄, filtered and concentrated in vacuo. Purification by flash chromatography (Hex/EtOAc 100:0 to 80:20) and crystallization from Hex gave the desired product (104 mg, 17%). LC (Zorbax, 30-100% CH₃CN): RT=3.425 min, LC/MS (ES+): 348, 350 [M+H].

The starting materials were prepared as described below:

### (5-Bromo-3-chloro-pyridin-2-yl)-(4-chloro-phenyl)-amine

A suspension of NaH (7.0 g, 175 mmol) in anhydrous THF (400 ml) was treated chloroaniline (22.5 g, 175 mmol) and then stirred for 1 h at rt. A solution of 5-bromo-2,3-dichloro-pyridine (20.0 g, 87.4 mmol) was added and the mixture was heated to reflux for 18 h. It was then poured onto a sat. aq. solution of Na₂CO₃ and the THF was evaporated. The aq. phase was extracted with EtOAc and the combined org. layers were then dried and concentrated in vacuo. Purification by flash chromatography (Hex/EtOAc 9:1) and crystallization from Hex/EtOAc afforded (5-bromo-3-chloro-pyridin-2-yl)-(4-chloro-phenyl)-amine (27.8 g, 66%). LC/MS (ES+): 319 [M+H].

### (3-Chloro-5-pent-1-ynyl-pyridin-2-yl)-(4-chloro-phenyl)-amine

A mixture of (5-bromo-3-chloro-pyridin-2-yl)-(4-chloro-phenyl)-amine (1.0 g, 3.14 mmol), 1-pentyne (624 μl, 6.29 mmol), [(PPh₃)₂PdCl₂] (113 mg, 0.16 mmol), CuI (15.3 mg, 0.08 mmol), and triethylamine (657 μl, 4.72 mmol) in DMF was heated to 100° C. for 24 h in a sealed tube. The mixture was allowed to cool to rt, then diluted with EtOAc, washed with water, dried over Na₂SO₄, filtered and concentrated in vacuo. Purification by flash chromatography (Hex/EtOAc 19:1) gave (3-chloro-5-pent-1-ynyl-pyridin-2-yl)-(4-chloro-phenyl)-amine (558 mg, 58%). LC/MS (ES+): 306 [M+H].

### EXAMPLE 47

### [3-Chloro-5-(2-isopropyl-imidazol-1-yl)-pyridin-2-yl]-(4-chloro-phenyl)-amine

A suspension of (5-Bromo-3-chloro-pyridin-2-yl)-(4-chloro-phenyl)-amine (200 mg, 0.63 mmol), 2-iso-propylimidazole (85 mg, 0.75 mmol), salicylaldoxime (18 mg, 0.13 mmol), CuI (9 mg, 0.06 mmol) and cesium carbonate (414 mg, 1.26 mmol) in CH₃CN (10 ml) was heated to 180° C. for 8 h in a microwave oven. The solvent was then evaporated and the crude product was purified by flash chromatography (Hex/EtOAc 100:0 to 0:100) to give [3-chloro-5-(2-isopropyl-imidazol-1-yl)-pyridin-2-yl]-(4-chloro-phenyl)-amine (46 mg, 21%). UPLC (5-100% CH₃CN): RT=1.244 min, MS (ES+): 347 [M⁺]

Following the same procedures, the following compounds can be obtained:

### EXAMPLE 48

### [3-Chloro-5-(5-methyl-imidazol-1-yl)-pyridin-2-yl]-(4-chloro-phenyl)-amine

MS (ES+): 319 [M⁺]

UPLC (5-100% CH₃CN): RT=1.148 min

### EXAMPLE 49

### [3-Chloro-5-(4-methyl-imidazol-1-yl)-pyridin-2-yl]-(4-chloro-phenyl)-amine

MS (ES+): 319 [M⁺]

UPLC (5-100% CH₃CN): RT=1.134 min

### EXAMPLE 50

### Biological Testing

Activity of compounds of the present invention was examined by measurement of the inhibition of the glutamate induced elevation of intracellular Ca²⁺-concentration following similar methods than those described in L. P. Daggett et al., Neuropharm. Vol. 34, pages 871-886 (1995), P. J. Flor et al., J. Neurochem. Vol. 67, pages 58-63 (1996).

The table below represents percentages of inhibition of the glutamate induced elevation of intracellular Ca²⁺-concentration at a concentration of 10 μM.

| Compound | mGluR5 Activity |
|---|---|
| Number | inh. at 10 μM [%] |
| 1 | 94 |
| 2 | 81 |
| 3 | 96 |
| 4 | 93 |
| 5 | 95 |
| 6 | 96 |
| 7 | 27 |
| 8 | 36 |
| 9 | 63 |
| 10 | 73 |
| 11 | 56 |
| 12 | 40 |
| 13 | 95 |
| 14 | 28 |
| 15 | 100 |
| 16 | 32 |
| 17 | 95 |
| 18 | 95 |
| 19 | 97 |
| 20 | 92 |
| 21 | 56 |
| 22 | 49 |
| 23 | 53 |
| 24 | 89 |
| 25 | 93 |
| 26 | 98 |
| 27 | 100 |
| 28 | 31 |
| 29 | 39 |
| 30 | 98 |
| 31 | 48 |
| 32 | 95 |
| 33 | 100 |
| 34 | 100 |
| 35 | 100 |
| 36 | 78 |
| 37 | 95 |
| 38 | 86 |
| 39 | 97 |
| 40 | 100 |
| 41 | 79 |
| 42 | 94 |
| 43 | 37 |
| 44 | 98 |
| 45 | 89 |
| 46 | 68 |
| 47 | 34 |
| 48 | 32 |
| 49 | 35 |

1. A compound defined by the formula 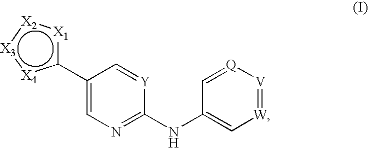
wherein
X₁, X₂, X₃, and X₄ are independently selected from the group consisting of CR¹, CO, N, NR², O and S,
R¹ and R² are independently selected from the group consisting of H, alkyl, substituted alkyl, benzyl, substituted benzyl, phenyl and substituted phenyl, or R₁ and R₂ form together with the atoms to which they are attached a hydrocarboncycle, a substituted hydrocarboncycle, a heterocycle or a substituted heterocycle,
Y represents CH or CR³ or N
V represents CH, CR⁴ or N
Q represents CH, CR⁵ or N
W represents CH, CR⁶ or N, and
R³, R⁴, R⁵, and R⁶ are independently selected from the group consisting of OH, halogen, alkyl, trifluoralkyl, alkoxy, trifluoralkoxy, and CN;
and pharmaceutically acceptable prodrugs, salts, solvates, hydrates, and N-oxides thereof.

2. The compound according to claim 1, wherein Y is CH or CCl.

3. The compound according to claim 1, wherein Q is CH or N.

4. The compound according to claim 1, wherein W is CH.

5. The compound according to claim 1, wherein V is CCl or CCH₃.

6. The compound according to claim 1, wherein one of the moieties X₁, X₂, X₃, and X₄ is N, another one of the moieties X₁, X₂, X₃, and X₄ is NR², a further one of the moieties X₁, X₂, X₃, and X₄ is CR¹ and the remaining one of the moieties X₁, X₂, X₃, and X₄ is either CH or N.

7. The compound according to claim 1, wherein X₁ is N.

8. The compound according to claim 1, wherein X₄ is NR².

9. The compound according to claim 1, wherein X₃ is CR¹.

10. The compound according to claim 1, wherein X₂ is CR¹ or N.

11. The compound according to claim 1, wherein X₁ is N, X₂ is CH, X₃ is CH or CCH₃, and X₄ is NR² with R² being a C₁ to C₄ alkyl, and optionally R₁ and R₂ form together with the atoms to which they are attached a six member ring.

12. The compound according to claim 1, wherein the compound is selected from the group consisting of 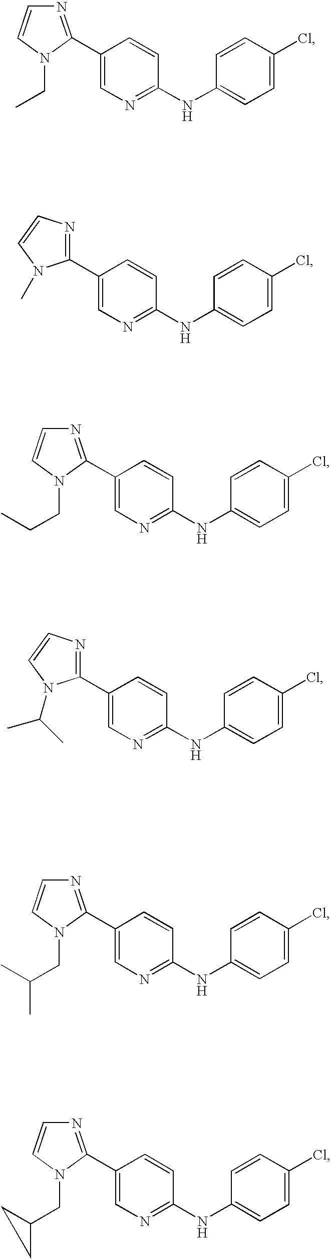 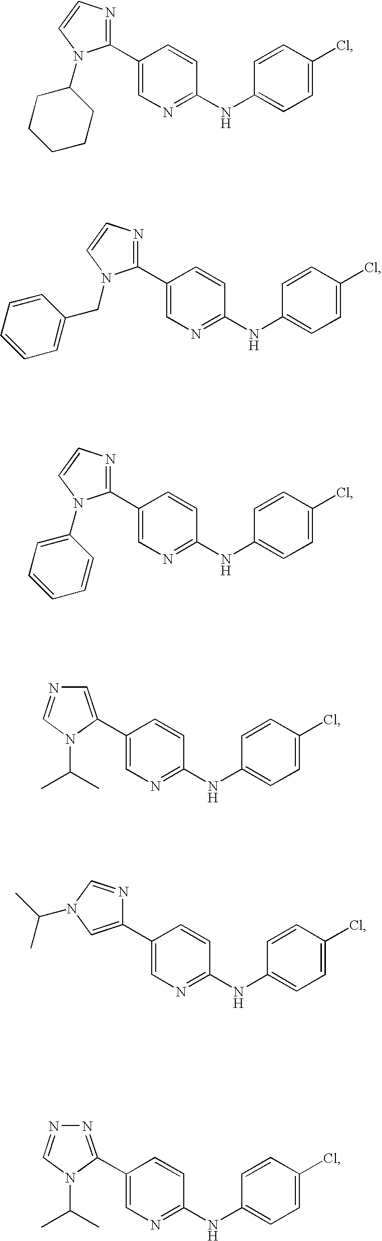 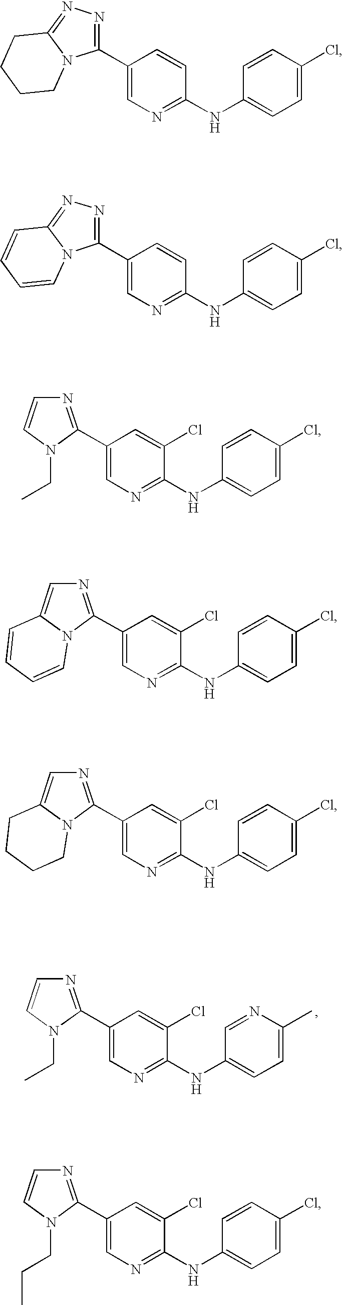 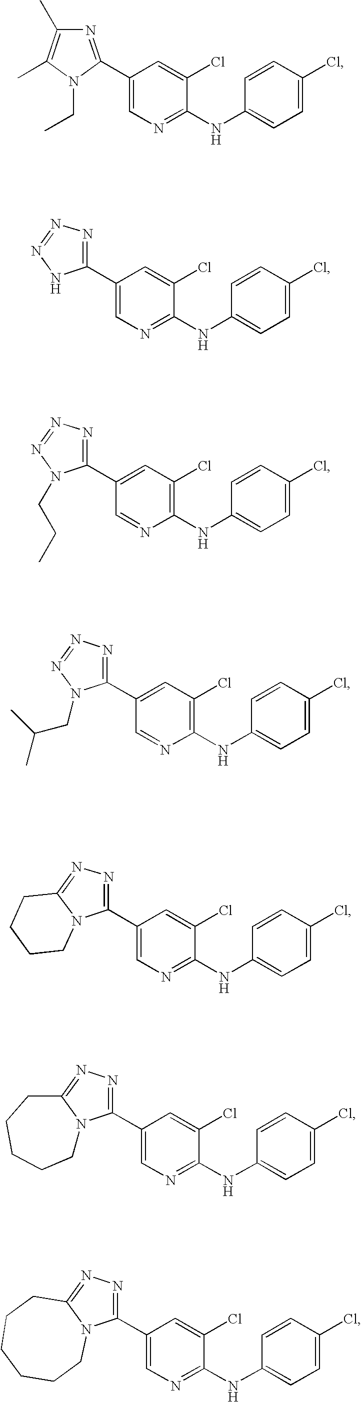 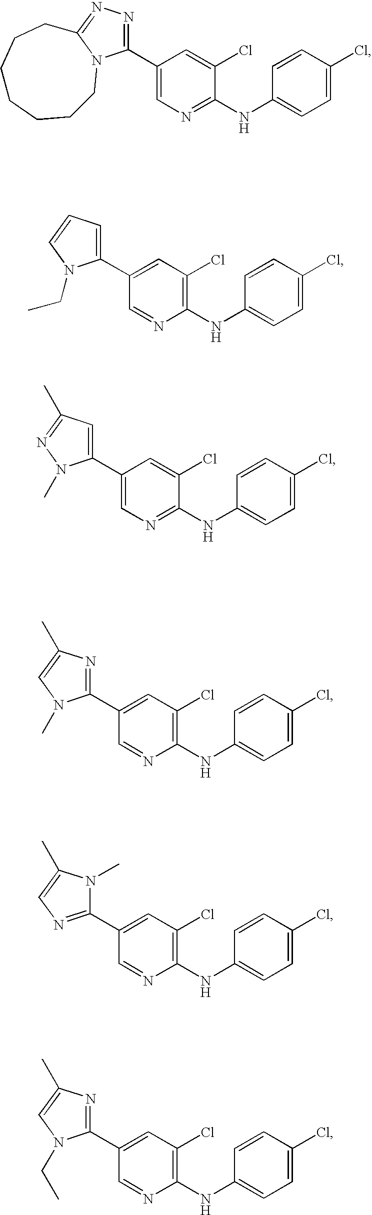 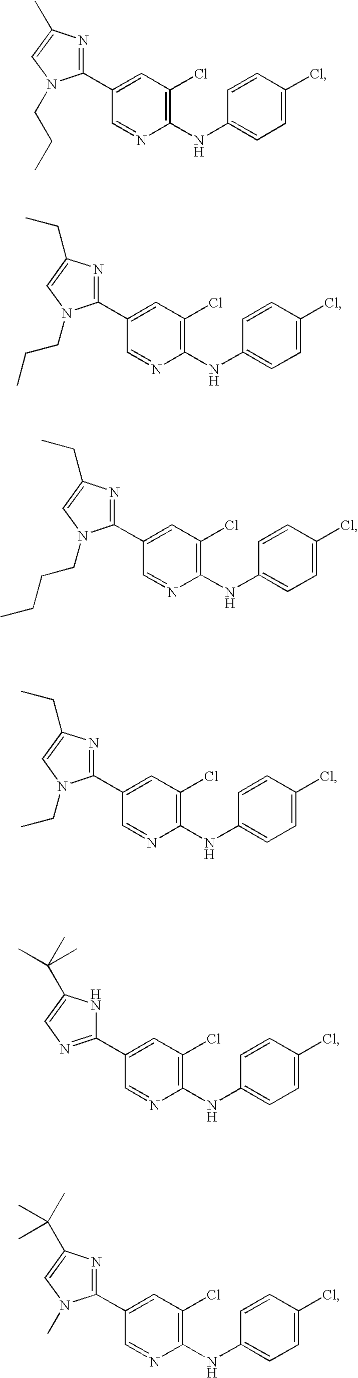 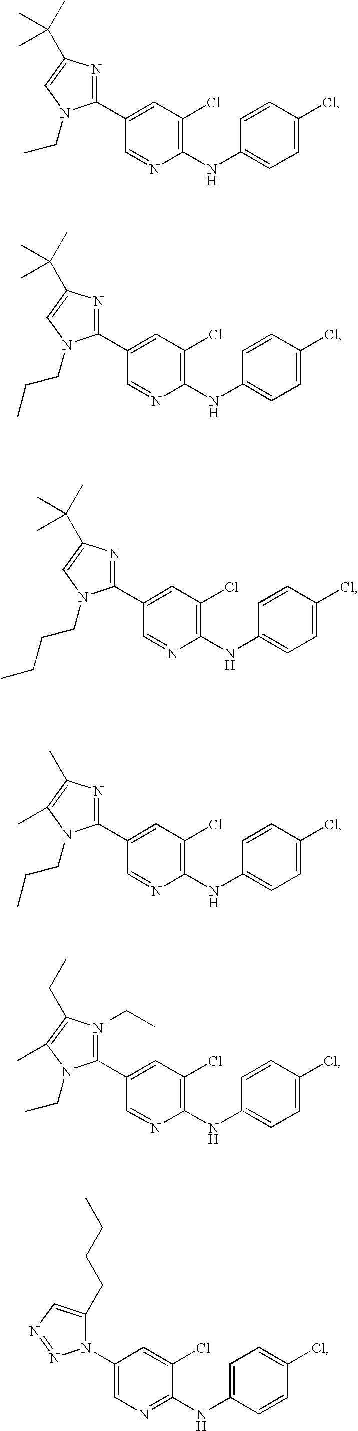 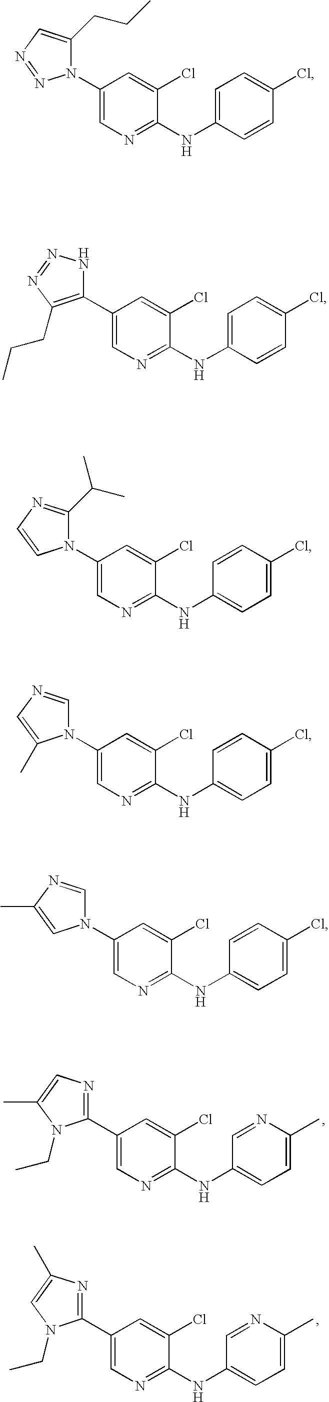 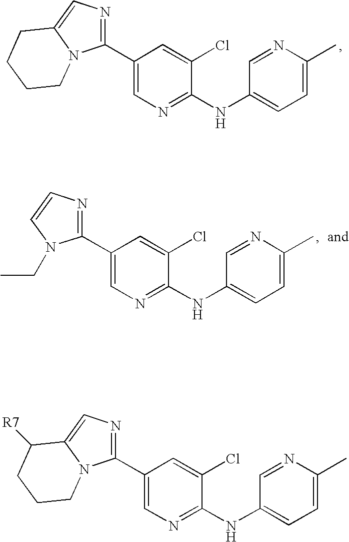
wherein R⁷ is alkyl or aryl.

13. The compound according to claim 1, wherein the compound is in free base or pharmaceutically acceptable acid addition salt form.

14. A process for the manufacture of the compound according to any one of the preceding claims, wherein the process comprises the step (A)

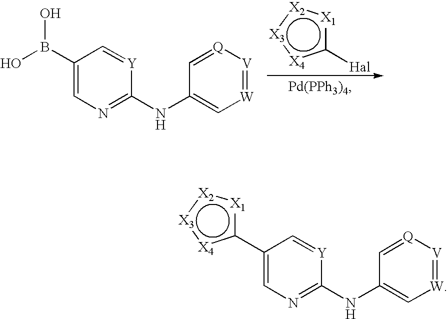

15. The process according to claim 14, wherein in the process additionally Na₂CO₃, methanol and inert solvent.

16. The process according to claim 14, wherein the process comprises the step (B) 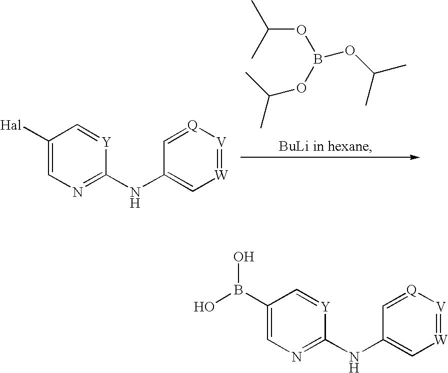
and wherein step (B) takes place in advance of step (A).

17. The process according to claim 14, wherein the process comprises the step (C) 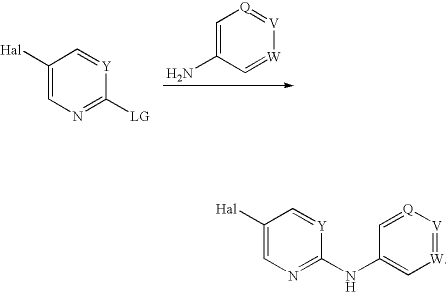
and wherein step (C) takes place in advance of step (A) or step (B).

18. The process according to claim 17, wherein the process comprises the steps (A), (B), (C) in the order of (C)→(B)→(A).

19. The process according to claim 1, wherein
Y is CH or CCl
Q is CH or N
W is CH
V is CCl or CCH₃, and
one of the moieties X₁, X₂, X₃, and X₄ is N, another one of the moieties X₁, X₂, X₃, and X₄ is NR², a further one of the moieties X₁, X₂, X₃, and X₄ is CR¹ and the remaining one of the moieties X₁, X₂, X₃, and X₄ is either CH or N.

20. A pharmaceutical composition, comprising:
the compound according to claim 1 and
a pharmaceutical carrier or diluent.

21-26. (canceled)
